(19) United States
(12) Patent Application Publication
(10) Pub. No.: US 2013/0295551 A1
(43) Pub. Date: Nov. 7, 2013

(54) MICROFLUIDIC DEVICE AND METHOD FOR MODULATING A GAS ENVIRONMENT OF CELL CULTURES AND TISSUES

(71) Applicant: THE BOARD OF TRUSTEES OF THE UNIVERSITY OF ILLINOIS, Urbana, IL (US)

(72) Inventors: David Eddington, Wheaton, IL (US); Gerardo Mauleon, Schaumburg, IL (US); Joe Fu-jiou Lo, Dearborn, MI (US); Megan Lynn Rexius, Chicago, IL (US); Jalees Rehman, Willowbrook, IL (US)

(21) Appl. No.: 13/840,570

(22) Filed: Mar. 15, 2013

Related U.S. Application Data
(60) Provisional application No. 61/617,189, filed on Mar. 29, 2012.

(51) Int. Cl. A01N 1/02; C12N 5/071

(52) U.S. Cl. CPC A01N 1/0247; C12N 5/0676
USPC 435/1.2; 435/297.2; 435/284.1; 435/374

(57) ABSTRACT

A microfluidic device having a perfusion chamber, the perfusion chamber having a base, a bath opening in the base, a supply inlet and an exhaust outlet. The device further includes a gas permeable membrane attached beneath the perfusion chamber, the gas permeable membrane having a first opening in registration with the supply inlet and a second opening in registration with the exhaust outlet. A substrate is attached to the gas permeable membrane, the substrate having at least one microchannel arranged for flow communication with the supply inlet and the exhaust outlet. In addition, a slide is attached to the substrate. As such, gas introduced through the supply inlet is communicated to the microchannel via the first opening, and the gas permeable membrane is positioned to be exposed to the gas to communicate the gas to the bath opening.

### STATEMENT OF U.S. GOVERNMENT SUPPORT

This invention was made with U.S. government support under Grant No. R21MH-085073 awarded by the National Institutes of Health, and Grant No. DBI-085214 awarded by the National Science Foundation. The government has certain rights in the invention.

### FIELD OF THE DISCLOSURE

This disclosure relates to a device and method for modulating a gas environment for cell cultures and tissues, and, more specifically, to microfluidic devices for controlling the oxygen environment of cell cultures and tissues.

### BACKGROUND OF THE DISCLOSURE

There is a need for improved methods of oxygen control in the biomedical research community. Current tools to modulate oxygen over cell cultures are relatively crude and insufficient and have not changed much since the dawn of cell culture techniques, as explained in more detail below.

For example, acute brain slice preparation is an excellent model for studying the details of how neurons and neuronal tissue respond to a variety of different physiological conditions. However, open slice chambers ideal for electrophysiological and imaging access have not allowed the precise spatiotemporal control of oxygen in a way that might realistically model stroke conditions, for example. More specifically, how neuronal tissue responds at the microscale to a hypoxic insult is a fundamental question for stroke research. The hippocampal acute brain slice preparation, with its defined cytoarchitecture, mechanical stability, and recognized sensitivity to oxygen variations, provides an in vitro model where the effect of oxygen deprivation on neuronal physiology can be studied in isolated detail. To fully understand the relationship between oxygen and neuronal function, one should subject the tissue to an environment where the oxygen supply can be controlled both temporally and on a spatial scale that mimics that in the living brain.

Most studies that subject isolated neuronal tissue to a hypoxic insult rely on perfusion chambers in which the oxygen is supplied by the perfusing liquid. The basic techniques used to supply oxygen to the slices have changed little since their conception, where the slice is placed in a perfusion chamber where artificial cerebral spinal fluid (aCSF) bubbled with 95% oxygen is perfused over the tissue. Depending on the chamber, the tissue can be completely submerged in the liquid or it can sit at the top of the fluid, with one side exposed to humidified air and the other exposed to the oxygenated perfusion. In order to expose the slice to a hypoxic environment, the oxygenated aCSF is switched to a deoxygenated aCSF (bubbled with 95% nitrogen) and in some cases sodium cyanide is applied to a small area of the tissue with the use of a pipette.

Unfortunately, perfusion-driven oxygen delivery is not controlled enough to oxygenate the slice homogeneously; oxygen gradients form throughout the slice with the core of the slice being hypoxic compared to the edges. Moreover, the delivery of oxygen to the brain slice cannot be precisely controlled and is cumbersome to isolate from any experimental chemicals that may be dissolved in the aCSF. Importantly, perfusion under typical protocols is all or nothing. It is impossible to selectively control oxygen levels on a scale that is spatially and temporally relevant to physiological ischemia.

In another example, simultaneous stimulation of ex vivo pancreatic islets, i.e., clusters of pancreatic cells, with dynamic oxygen and glucose loads is critical to understand how hypoxia alters the glucose-insulin response, especially for transplant applications. However, standard techniques using a hypoxic chamber cannot provide both oxygen and glucose modulations while monitoring in real-time.

More specifically, ex vivo study of islets of Langerhans, 50-400 μm spheroidal aggregates of pancreatic endocrine cells, under controlled microenvironments is critical for studying islet physiology. In vivo, islets are highly perfused with their insulin secretion significantly influenced by the dynamics of blood flow, oxygen supply, and glucose gradients. Yet once the islets are isolated, their nutrient supply is limited to the first 100 μm of the islet due to diffusion limitations. Recreating the dynamic oxygen and glucose profiles is difficult with current experimental protocols, which require high flows of pressurized gas in hypoxic chambers and elaborate flow schemes. Moreover, when isolated islets are exposed to changing oxygen levels, such as transplants to the venous hepatic portal, their insulin secretion is compromised by hypoxia. Hypoxia, together with transplant size and immunosuppressive regiment, remain the three main challenges facing a promising islet therapy using the Edmonton Protocol for type I diabetes. To address islet hypoxia, developing a technique for dynamic oxygen, including intermittent hypoxia (IH), could drastically improve islet responses by preconditioning them before exposure to in vivo hypoxia levels.

Preconditioning effects, first reported in Murry et al 1986 in heart infarctions, is increasingly evident in ex vivo tissues. Mitigation of cardiac myocyte infarction was originally preconditioned by ischemic and later IH methods. Similarly, ischemia-reperfusion preconditioning improves kidney function after transplantation. Ischemic protection has also been shown against strokes in neural tissues. However, preconditioning has not been demonstrated in pancreatic islets, which are exposed to hypoxia during procurement and transplantation. Even several days after transplantation, the islets are supplied oxygen and nutrients solely by diffusion, and exposed to much lower PO₂ (3-5 mmHg) in the hepatic portal system, compared to physiological pancreas (40 mmHg), for up to two weeks until revascularization. Studies using current methods suggest that glucose stimulated insulin response (GSIR) can be impaired by hypoxia, but a real-time stimulation and monitoring technique is required to directly investigate this relationship. Among GSIR parameters, simultaneous calcium, mitochondria, and insulin responses can be easily monitored using microfluidics. To address the hypoxic damage to islets during isolation, oxygenated perfluorocarbon solution has been used during pancreas procurement, while ischemic and intermittent hypoxia preconditioning have been tried without success. Those dynamic hypoxia studies are complicated by the inefficient, pressurized, high-flow hypoxic chambers that lack real-time islet functional assessment. Nevertheless, strong evidence shows mitochondrial KATP (mitoKATP) openers used for cardiac myocyte preconditioning can benefit islets. Thus, simultaneous modulation of glucose and hypoxia, including transient dynamics at islets' microscaled-level, represents a critical technique to enable novel IH functional studies and achieve islet preconditioning.

In addition, and more generally, current devices used for modulating oxygen in vitro cultures, such as a hypoxic chamber, a segmented incubator, or sealed glove boxes have limitations. For example, such devices: 1) are prone to leaks; 2) have low throughput; 3) are unable to replicate anoxic conditions or physiologic O2 gradients; 4) require hours to equilibrate, 5) are incompatible with rapid microscopic analysis, and 6) have cumbersome setups. Several groups have developed devices aimed at improving the hypoxic chamber for controlling oxygen tensions in cell cultures. Despite advances in equilibration time and ability to set up limited gradients, many such devices require very specific parameters for operation, including the need for complex fluid handling, and in some cases electronic controls.

### SUMMARY OF THE DISCLOSURE

Described herein is a suite of devices to modulate dissolved gasses for mammalian cell cultures. These devices all leverage the innate gas permeability of polydimethylsiloxane (PDMS) and allow for precise microscale control over the oxygen environment of biological material, such as cell cultures.

In one example of the present disclosure, a microfluidic device comprises a perfusion chamber, the perfusion chamber having a base, a bath opening in the base, a supply inlet, and an exhaust outlet. The device further includes a gas permeable membrane attached beneath the perfusion chamber, the gas permeable membrane having a first opening in registration with the supply inlet and a second opening in registration with the exhaust outlet. A substrate is attached to the gas permeable membrane and has at least one microchannel arranged for flow communication with the supply inlet and the exhaust outlet, and a slide is attached to the substrate. A gas introduced through the supply inlet is communicated to the microchannel via the first opening, and the gas permeable membrane is positioned to be exposed to the gas to communicate the gas to the bath opening.

In addition, the bath opening may define an area to receive biological material, the biological material comprising one or more of a brain slice or cultured cells.

Also, a top surface of the gas permeable membrane may define an area to receive biological material, and the microfluidic channel substrate may include a plurality of separate flow channels or microchannels, at least some of the plurality of flow channels arranged to be selectively blocked. The biological material may be exposed to different concentrations of a gas communicated through the plurality of flow channels.

Further, one or both of the gas permeable membrane and the microfluidic channel substrate may be constructed polydimethylsiloxane (PDMS).

Still further, the thickness of the membrane may be about 50 μm to 200 μm.

Moreover the thickness of the microfluidic channel substrate may be about 50 μm to 100 μm.

In addition, the substrate may further include a support pillar that prevents the gas permeable membrane from collapsing.

In another example of the present disclosure, a microfluidic device for modulating a gas environment of a biological material comprises a microfluidic channel and a gas permeable membrane disposed on the microfluidic channel, the gas permeable membrane having a top surface and bottom surface. A chamber is disposed on the top surface of the gas permeable membrane, and a gasket is disposed on the chamber. A first supply port is connectable to a first supply source and disposed through each of the microfluidic channel, the membrane, the chamber, and the gasket, and a second supply port is connectable to a second supply source and disposed through each of the chamber and gasket. The microfluidic channel delivers a first medium from the first supply port directly to the bottom surface of the membrane disposed on the microfluidic channel. In addition, the chamber delivers a second medium from the second supply port directly to the top surface of the membrane, allowing simultaneous first and second media stimulation within the microfluidic device.

### BRIEF DESCRIPTION OF THE DRAWINGS

FIG. 1A is a perspective view of a perfusion device of a microfluidic device according to one aspect of the present disclosure;

FIG. 1B is a perspective, exploded view of the microfluidic device of according to one aspect of the present disclosure;

FIG. 1C is a perspective, assembled view of the microfluidic device of FIG. 1B;

FIGS. 2A and 2B illustrate oxygen concentration (%) as a function of time (min) for a diffusion device and traditional perfusion;

FIGS. 2C and 2D illustrate oxygen concentration (%) as a function of distance (μm) for the diffusion device, perfusion and a perfusion and diffusion device;

FIG. 3A illustrates percent change in oxygen as a function of time (min) for the microfluidic device of the present disclosure;

FIGS. 3B and 3C show magnification images showing cells before and during a hypoxia or oxygen period;

FIG. 3D illustrates another graph showing percent change as a function of time (min) for the microfluidic device of the present disclosure;

FIG. 4A is a perspective, magnified view of a portion of another embodiment of a microfluidic device that may also include the perfusion chamber of FIG. 1A, the view illustrating a changing microfluidic channel and a constant microfluidic channel;

FIG. 4B is a magnified image of an area of portion of the microfluidic device of FIG. 4A illustrating two channels, the changing and the constant microfluidic channels;

FIG. 4C illustrates percent change in oxygen and Fura as a function of distance (mm) for the device of the present disclosure;

FIG. 5A illustrates a percent change in oxygen concentration as a function of time (min) for both a changing and a constant microfluidic channel of the microfluidic device of FIG. 4A;

FIGS. 5B-5E show portions of magnified images of cells disposed on the membrane of the device of the present disclosure during various conditions, e.g., initial conditions, hypoxis conditions, and no change in oxygen conditions;

FIG. 6A illustrates another example of microfluidic device of another aspect of the present disclosure;

FIG. 6B illustrates a system into which the microfluidic device of FIG. 6A may operate;

FIG. 6C is a chart illustrating simultaneous control of oxygen, flow and glucose stimulation at cells in the microfluidic device of FIG. 6A;

FIG. 6D is shows portions of magnified images of cells within the microfluidic device of FIG. 6A with multiple staining;

FIGS. 7A and 7B illustrate oxygen and FITC intensity as a function of time (min) for the microfluidic device of FIG. 6A and delivered and dissolved oxygen as a function of time (min) for the microfluidic device of FIG. 6A, respectively;

FIG. 8A illustrates the FURA ratio as a function of time (min) for biological material of the microfluidic device of FIG. 6A;

FIGS. 8B and 8C illustrate the FURA ratio percentage as a function of time (min) for the biological material of the microfluidic device of FIG. 6A;

FIG. 9A illustrates another FURA ratio as a function of time (min) for the biological material of the microfluidic device of the FIG. 6A;

FIGS. 9B and 9C illustrate another FURA ratio percentage as a function of cycle time (min) and frequency, respectively, for the biological material of the microfluidic device of FIG. 6A;

FIG. 10A illustrates insulin as a function of time (min) for the biological material of the microfluidic device of FIG. 6A;

FIG. 10B illustrates an insulin secretion index and relative calcium and mitochondrial changes for the biological material of the device of FIG. 6A;

FIG. 11A illustrates a front, cross-sectional view of yet another example of a microfluidic device according to another aspect of the present disclosure;

FIG. 11B illustrates a top perspective view of the microfluidic device of FIG. 11A;

FIG. 11C shows magnified images of cells under hypoxia and normoxia in the microfluidic device of FIG. 11A, respectively;

FIG. 11D is a graph illustrating higher levels of relative gene expression in hypoxic regions of the microfluidic device of FIG. 11A;

FIG. 12A illustrates a front, cross-sectional view of yet another example microfluidic device according to another aspect of the present disclosure;

FIG. 12B illustrates a top perspective view of the microfluidic device of FIG. 12A;

FIGS. 12C and 12D show magnified images of cells under hypoxia in one region and normoxia in another region of the microfluidic device of FIG. 12A;

FIG. 12E is a graph illustrating higher levels of relative gene expression in hypoxia regions and dual condition of the microfluidic device of FIG. 12A;

FIG. 13A illustrates a perspective view of the microfluidic device of FIG. 12A;

FIG. 13B illustrates a top view of the microfluidic device of FIG. 13A;

FIG. 13C illustrates a portion of a section of a microfluidic channel of the microfluidic device of FIG. 13A;

FIG. 14 illustrates a prior art device using a standard method;

FIG. 15A is a front, cross-sectional view of yet another microfluidic device according to another aspect of the present disclosure;

FIG. 15B illustrates oxygen concentration (%) as a function of time (min) under both the standard method of FIG. 14 and for the microfluidic device of FIG. 15A;

FIG. 15C is a graph illustrating the percentage of live cells as a function of mice age (days) for both the standard method of FIG. 14 and the microfluidic device of FIG. 15A;

FIG. 15D is a graph illustrating the percentage of loaded cells per area (cells/mm 2) as a function of mice age (days) for both the standard method of FIG. 14A and the microfluidic device of FIG. 15A;

FIG. 15E is another graph illustrating the intracellular calcium concentration (% change) as a function of time for the biological material of the microfluidic device of FIG. 15A;

FIGS. 15F and 15G show various magnified images of a portion of the biological material increasing in time, e.g., P12, P20, P30, for both the standard method of FIG. 14 and the microfluidic device of FIG. 15A; and

FIG. 15H is a table showing loading time (min), viability improvement (fold), and loading improvement (fold) for various ages of the biological material in the microfluidic device of FIG. 15A.

### DETAILED DESCRIPTION OF THE DISCLOSURE

Although the following text sets forth a detailed description of exemplary embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology developed after the filing date of this patent, with those alternative embodiments still falling within the scope of the claims defining the invention.

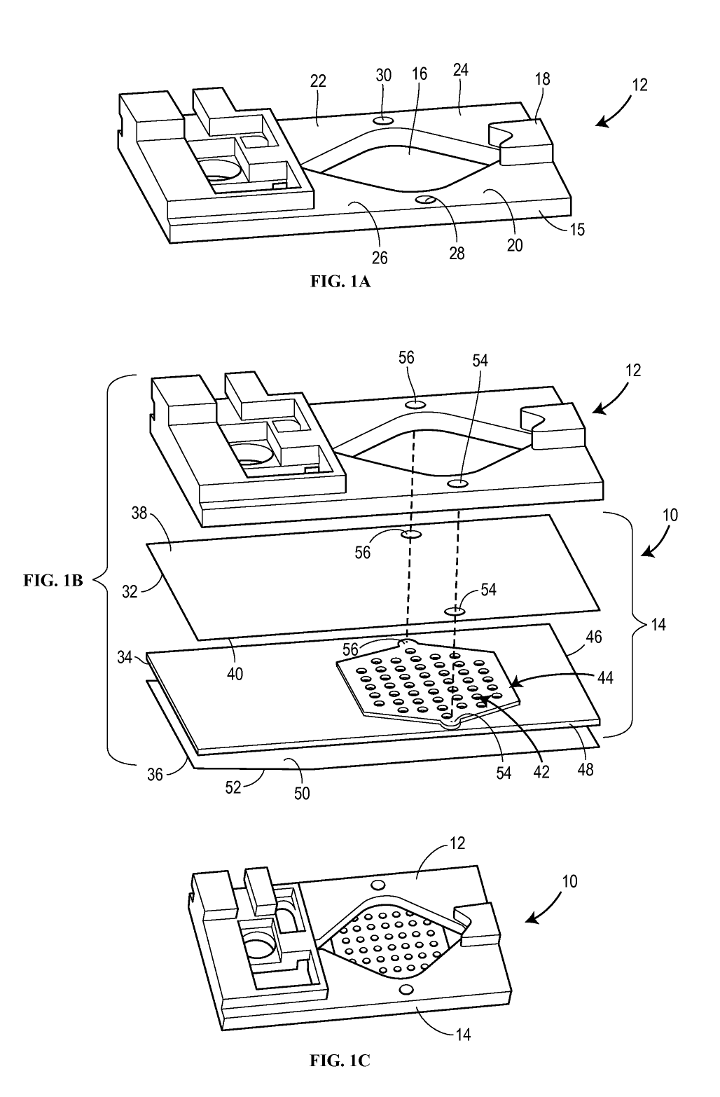

Referring now to FIGS. 1A-1C, a microfluidic device constructed in accordance with the teachings of an example of the present invention is shown and is identified generally by the reference numeral 10. The device 10 includes a perfusion chamber 12 and a microfluidic device lower portion 14. More specifically, as illustrated in FIG. 1A, the perfusion chamber 12 includes a base 15 and a bath opening 16 disposed in the base 15 and for receiving biological material, such as a brain slice. The perfusion chamber 12 also includes a perfusion inlet 18 that is disposed on the base 15, a front end 20, a rear end 22, a top surface 24 and a bottom surface 26. Two holes were drilled into the base 15 of the perfusion chamber 12 to act as a gas inlet and a gas outlet. More specifically, in one example, a gas or supply inlet or port 28 is disposed on a front end 20 of the perfusion chamber, and a gas or exhaust outlet or port 30 is disposed on a rear end 22 of the perfusion chamber 12. As one of skill in the art will appreciate, the perfusion chamber 12 may be a standard perfusion chamber (RC-26GLP).

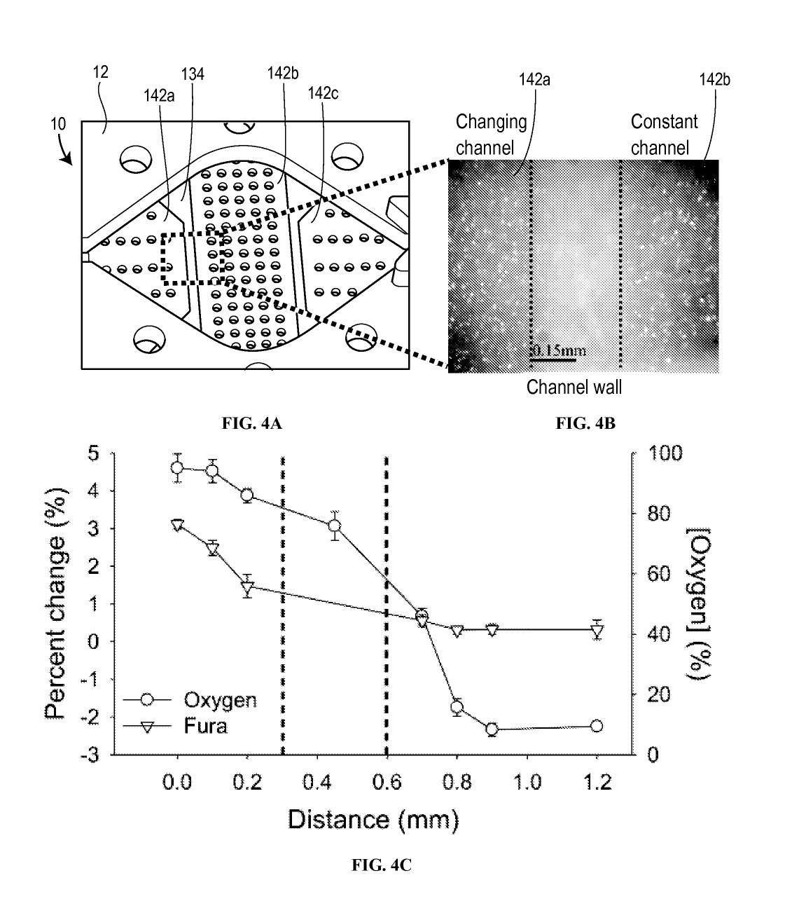

As illustrated in FIG. 1B, the microfluidic device 10 includes a gas permeable membrane 32, a microfluidic channel substrate 34 disposed below the gas permeable membrane 32, and a slide 36, such as a glass slide, disposed below the microfluidic channel substrate 34. The gas permeable membrane has a top surface 38 and a bottom surface 40. The microfluidic channel substrate 34 includes at least one microchannel 42 for delivering gas directly to the gas permeable membrane 32 and may alternatively include more than one channel or a plurality of channels or flow channels, as illustrated in FIG. 4A, for example and explained in more detail below. The microfluidic channel substrate 34 also includes a support pillar 44 that prevents the gas permeable membrane 32 from collapsing and, thus, blocking the microfluidic channel substrate 34. The microfluidic channel substrate 34 further includes a top surface 46 that contacts and is bonded to the bottom surface 40 of the gas permeable membrane 32 once assembled, and a bottom surface 48. The slide 36 likewise includes a top surface 50 that contacts and is bonded to the bottom surface 48 of the microfluidic channel substrate 34, and a bottom surface 52. The slide 36 may comprise glass or various other transparent or translucent materials that enable viewing therethrough by a microscope disposed beneath the glass slide, as explained in more detail below. The slide 36 provides further support to the microfluidic channel substrate 34 attached thereto. Each of the gas permeable membrane 32, the microfluidic channel substrate 34 and the slide 36 includes a supply or first opening 54 that is in registration with the supply inlet 28 and an exhaust or second opening 56 that is in registration with the exhaust outlet 30 of the perfusion chamber 12.

To assemble the microfluidic device lower portion 14, the top surface 50 of the slide 36 is attached and bonded to the bottom surface 48 of the microfluidic channel substrate 34, and the bottom surface 40 of the gas permeable membrane 32 is attached and bonded to the top surface 46 of the microfluidic channel substrate 34. The bonding method and process is explained in much greater detail below. More generally, the microfluidic channel substrate 34 is sandwiched between the gas permeable membrane 32 on the top surface 46 and the slide 36 on the bottom surface 48, forming the microfluidic device lower portion 14. The bottom surface 26 of the perfusion chamber 12 is then attached and bonded to the top surface 38 of the gas permeable membrane 32, forming the microfluidic device 10, as illustrated in FIG. 1C.

The gas permeable membrane 32 is constructed of polydimethylsiloxane or PDMS, as also explained in more detail below. In addition, in one example, the microfluidic channel substrate 34 is also constructed of PDMS, but one of skill in the art will appreciate that this material is not required for the microfluidic channel substrate 34 to operate effectively. As such, other materials may be used in the construction of the microfluidic channel substrate 34 and still fall within the scope of the present disclosure.

In accordance with a preferred embodiment, a thickness of the membrane 32 may be about 100 μm, although a broader range of about 50 to about 200 μm may suffice. Other thicknesses may prove suitable. The thickness may be chosen to balance the benefit of rapid gas transport with the limitation of increased deflection under applied pressures with thinner membranes. For example, a membrane having a thickness less than 100 μm may tear when exposed to certain pressures, or may deform beyond acceptable or desired limits. A membrane having a thickness too great may inhibit gas permeation through the material, which may lead to uncertainty regarding the actual gas concentration exposure within the well.

The microfluidic device lower portion 14 is an add-on to the commercially available perfusion chamber 12 and diffuses oxygen throughout the gas permeable membrane 32 and directly to the brain slice or cell cultures disposed thereon. The microfluidic channel substrate 34 enables rapid and efficient control and delivery of oxygen and can be modified to allow different regions of the slice to experience different oxygen conditions or environments. Using this novel device 10, a stable and homogeneous oxygen environment throughout the brain slice is achieved and the oxygen tension in a hippocampal slice may be rapidly altered. In addition, and as explained in more detail below, different oxygen tensions may be imposed on different regions of the slice preparation and two independent responses measured, which is not easily obtainable with current techniques.

Using this device 10, the brain slice is in direct contact with the gas permeable membrane 32 with oxygen gas via at least one microchannel 42 of the microfluidic channel substrate 34 disposed beneath the membrane 32. This allows leverage of rapid microscale diffusion to achieve a more stable and uniform oxygen environment throughout the brain slice than is possible with only perfusion using only a perfusion chamber 12. Finally, independently oxygenation of different regions of the hippocampus and two independent responses are measured, demonstrating the utility to stroke research and neuroscience in general. Because a commercially available open bath perfusion chamber 12 is being modified, this technology can be used alongside standard electrophysiology tools.

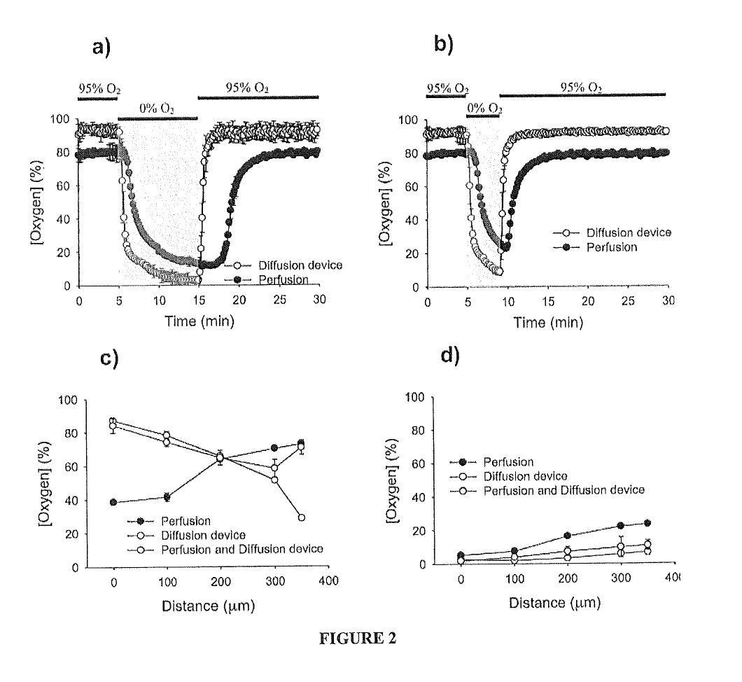

Referring now to FIGS. 2A-2D, various graphs illustrate the validation of the microfluidic device 14 of the FIGS. 1B and 1C with an oxygen sensor (not shown). More specifically, FIGS. 2A and 2B show the oxygen concentration dissolved in the aCSF that was measured for the microfluidic or diffusion device 10 and traditional perfusion using an oxygen probe (Neofox, Ocean Optics) and a transient hypoxic stimulus lasting 10 and 4 minutes. No slice was present during these experiments. The microfluidic or diffusion device 10 obtains a more controlled as well as a bigger change in oxygen concentration. As illustrated in FIG. 2C, using the oxygen sensor mounted to an electric translation manipulator, the oxygenation at depths of 0, 100, 200, 300, and 350 μm were measured, with 0 μm marking the bottom of the chamber. Perfusion and device 10 measurements are recorded. The graph shows the oxygen concentration during the oxygenation period (95% O₂). The graph of FIG. 2D shows the oxygen concentration inside the tissue obtained at the end of the hypoxic stimulus.

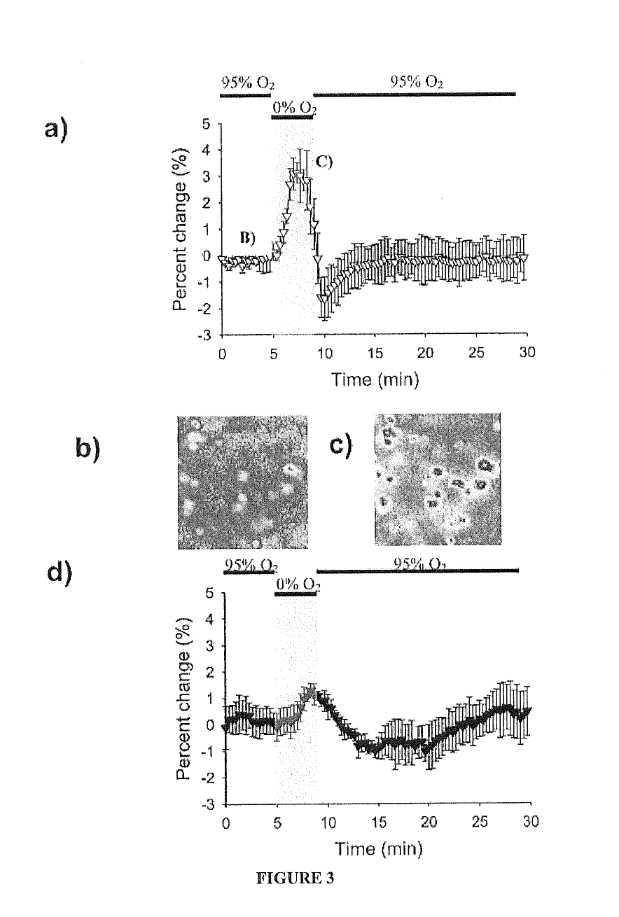

Referring now to FIGS. 3A-3D, Fura readings for the microfluidic or diffusion device 14 and the perfusion method are provided. The graph of FIG. 2A shows the Fura signal obtained by the microfluidic device 10. Increases in signal intensity signify an increase in intracellular calcium. The hypoxic period is represented by the gray rectangle. The 10× magnification images of FIGS. 2B and 2C show CA1 cells before and during the hypoxia period. The increase in fluorescence in FIG. 2C corresponds to an increase in intracellular calcium as previously mentioned. The graph of FIG. 2D shows the Fura signal obtained by the perfusion method.

Referring now to FIGS. 4A-4C, another embodiment of the microfluidic device 10 of FIGS. 1A-1C is depicted that provides not only temporal control, but spatial control as well. This second device is essentially identical to the previous device except that instead of one microfluidic channel 42 (FIG. 1B) supplying the oxygen to the entire brain slice, several independent microchannels 142a, 142b, 142c (FIG. 4A) can supply different regions of the slice with different oxygen concentrations. As with the previous device, this device lower portion docks with a common perfusion chamber 12. More specifically, FIG. 4A is a magnified view of a portion of the device 10 (FIGS. 1A-1C) used to oxygenate different regions of the brain slice disposed on the gas permeable membrane 32 of the microfluidic device 10. The square shows the location where the measurements for FIG. 4C were taken. A 10× magnified image showing the two channels used for data collection with the 0.3 mm wall at the center of the image is illustrated in FIG. 4B, as explained in more detail below. Data from both sides of the image were collected. To test the spatial resolution of the device, the oxygen and Fura measurements were taken at fixed positions across the plurality of microchannels 142 of the microfluidic channel substrate 34 (FIG. 1B), as illustrated in the graph of FIG. 4C. During the experiments, one channel of the plurality of microchannels 142 is flowing 95% oxygen (O₂) gas, while an adjacent channel of the plurality of microchannels is flowing 0% oxygen (O₂) gas. As shown in the graph, there is a steep change, in both oxygen and Fura, in the measurements obtained from the two adjacent channels. Dashed lines indicate the channel wall's boundaries.

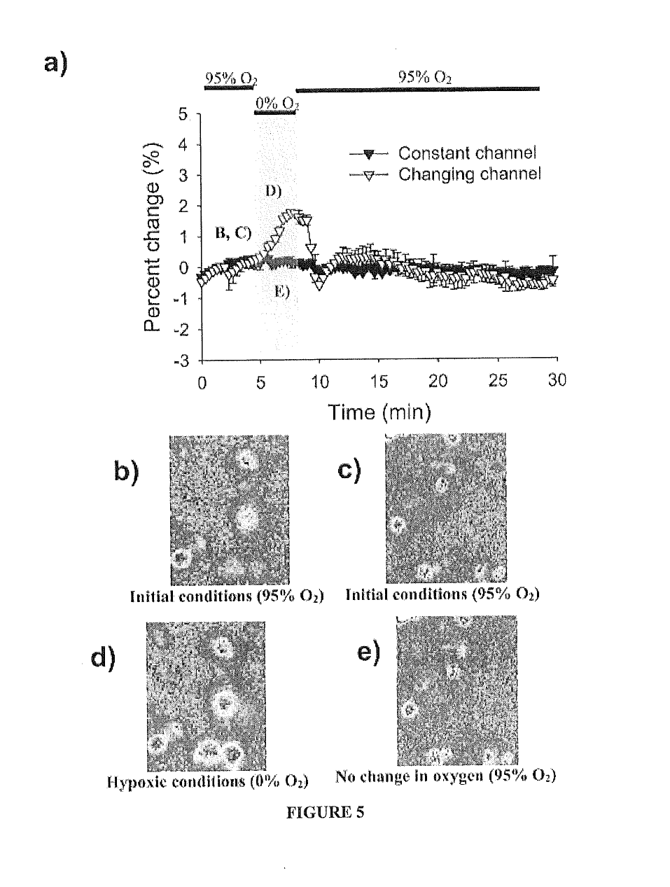

Referring now to FIGS. 5A-5E, Fura readings from two different hippocampal regions of a brain slice disposed within the device 10 of FIGS. 4A-4C are illustrated. As indicated in FIGS. 4A-4C, for example, the device 10 is able to maintain 2 different oxygen concentrations in different parts of the same brain slice using a microfluidic channel having two microchannels. In FIGS. 5A-5E, the brain slice was positioned in such a way as to have the CA1 in one microchannel and the dentate gyrus in a second microchannel. The CA1 area was exposed to a hypoxic insult while the dentate gyrus was kept to a constant oxygen environment. FIGS. 5B and 5D are 10×-magnification images demonstrating the increase in intracellular calcium experienced by the CA1 area. FIGS. 5C and 5E show 10× magnification images showing the dentate gyrus did not suffer a change in calcium concentration, as expected.

### Materials and Methods

Design of Oxygen Delivery Add-on:

The microfluidic channel substrate 34 and the membrane 32 were fabricated out of the elastomer polydimethylsiloxane (PDMS) using soft lithography as previously described. Alignment marks were used to create holes in the PDMS membrane 32 and in the perfusion chamber 12 in such a way that they allowed the oxygen to flow into and out of the microfluidic channel 34 below the gas permeable PDMS membrane 32. Once the individual parts are aligned, the parts are irreversibly bonded to complete the device 10. The oxygen gas is supplied at a rate of 38 ccm to the microfluidic gas channel 34 to allow adequate perfusion through the microfluidic channel substrate 34 without distending the membrane 32. The perfusion chamber 12 allows the slice to be completely submerged under the aCSF while the PDMS membrane 32 provides a mechanically stable surface for the tissue. The optically transparent PDMS allows clear visualization of the neurons from the bottom surface 52 of the microfluidic device 10, which is necessary to measure changes in fluorescence intensity observed with the calcium indicator using an inverted microscope. Even better results should be expected when using immersion objectives and upright microscopes typically used for electrophysiology studies on slice preparations. Also, an open bath perfusion chamber 12 is being modified, and the top surface 24 of the perfusion chamber 12, which forms a top surface of the device 10, allows use of all applicable neuroscience tools associated with open bath setups.

Fabrication of Oxygen Delivery Add-on:

The oxygen delivery add-on device consists of two polymeric parts: the microfluidic network or microfluidic channel 34 and the gas-permeable membrane 32. The photomask design was designed in Adobe Illustrator CS4 and printed on high-resolution (16,000 dpi) transparency film (Fineline Imaging, Colorado Springs, Colo.). The design consisted of either a single or multiple large gas chambers with 500 μm cylindrical support pillars 44 to prevent the membrane 32 from distending or collapsing. To fabricate the negative mold master, a 3 in silicon wafer was thoroughly cleaned before being exposed to oxygen plasma (Plasmatic Systems, Inc. Plasma-Preen II-862, North Brunswick, N.J.). Next, the treated wafer was spin-coated (Laurel) with SU-8 2100 photoresist (MicroChem Corporation, MA) to achieve a thickness of 200 μm (spun at 1500 rpm for 30 sec). This wafer/master is then soft baked (95° C. for 40 minutes), selectively exposed (315 mJ/cm²), post-exposure baked (95° C. for 14 minutes), and developed. To fabricate the positive mold of the device, 2 batches of 5 grams of polydimethylsiloxane (PDMS) (Sylgard 184 kit, Dow Corning) were prepared (10:1 mixture of prepolymer and curing agent; degassed under vacuum). First, one of the PDMS batches was spin-coated on the master to achieve a thickness of 100 μm (spun at 800 rpm for 30 sec) followed by curing for 15 minutes at 75° C. Then, the second batch of PDMS is spin-coated on top of the master at the same speed followed by curing for 2 hours at 75° C.; this combined process creates a uniform 200 μm thick PDMS layer. Once the PDMS layer is cured, it can be separated from the master mold and bonded to one 22×40 mm coverglass using oxygen plasma exposure (Plasmatic Systems, Inc. Plasma-Preen II-862, North Brunswick, N.J.).

To make the gas-permeable membrane 32, 5 grams of PDMS was mixed as mentioned above. Next, the PDMS was spin-coated on a new silicon wafer to achieve a thickness of 100 μm followed by curing for 2 hours at 75° C. Once the PDMS layer was cured, a section that would fit the microfluidic network was removed from the wafer and placed on a transparency film. Following this step, using alignment marks, the inlet and outlet ports 54, 56 were made in the membrane 32 using a blunted punch hole. Once the gas-permeable membrane 32 and the microfluidic channel 34 or network were ready, they were exposed to oxygen plasma and bonded together, making sure that the holes 54, 56 in the membrane would make contact with the inlet and outlet 54, 56 of the microfluidic channel substrate 34.

Standard Perfusion Chamber Attachment:

This process is similar to what has been previously described. Briefly, inlet and outlet ports 28, 30 were drilled in opposite sides of the standard perfusion chamber 14 (RC-26GPL, Warner Instruments) making sure that the ports 28, 30 would properly align with the oxygen delivery device (not shown). To bond the perfusion chamber to the membrane, a light coating of PDMS was applied to the bottom surface 26 of the perfusion chamber 14 as an adhesive.

Validation of Device Using Hand-Held Optical Sensor:

A hand-held optical sensor (Neofox, Ocean optics) was used to determine the oxygen concentration inside the brain slice. The sensor (not shown) was calibrated according to the manufacturer's instructions, namely, 95% N₂/5% CO₂ and 95% O₂/5% CO₂ was used to represent 0% O₂ and 95% O₂ respectively as CO₂ can also alter the fluorescence of the probe. The oxygen concentration inside the brain slice—the hippocampal CA1 area—was gathered in three different steps. First, two flasks with aCSF solution were bubbled with 95% N₂/5% CO₂ and 95% O₂/5% CO₂ until the aCSF's oxygen concentrations was 3±2% O₂ and 91±2% O₂ as measured with the optical sensor. Next, while in a standard perfusion chamber 12, the oxygen concentration inside the brain slice was measured while cyclic oxygenated and deoxygenated flows were applied—all experiments involving perfusion were done at a rate of 2 ml/min unless otherwise indicated. Following that test, a brain slice was placed inside the finalized oxygen delivery device 10. Then, the chamber 12 was filled with aCSF (no flow for this experiment), and the oxygen concentration inside the brain slice was measured while different oxygen gasses (0%, 95% O₂) were injected through the device 10. As a third test, the oxygen concentration inside the brain slice was measured while the gas was injected through the microchannels 42 and aCSF was perfusing through the chamber 12 (combination of oxygenation methods).

In order to measure the oxygen concentrations inside the brain slice, the oxygen probe was attached to an electronic manipulator that could maneuver the probe in the x, y, and z planes with a resolution of 0.1 μm. For our purposes, the oxygen concentration at a height (starting from the bottom of the chamber) of 0, 100, 200, 300, and 350 μm (top of the slice) were measured. The oxygen concentrations of the aCSF during the experiments were also measured.

Brain Slice Preparation:

The Animal Care and Use Committee at the University of Illinois Chicago approved all of the procedures outlined here. Post-natal 24 days wild type BL7 mice were deeply anesthetized using Aerrane (isoflurane, USP) and decapitated. Brains were rapidly removed from the skull and placed in chilled (3-7° C.) high-sucrose cutting solution. Then, the cerebellum was separated and disposed, while the rest of the brain tissue was glued to an agar block using superglue with the cerebral cortex facing down. Next, while in high-sucrose cutting solution, 350 μm thick hippocampal slices were cut with a tissue slicer (Vibratome Series 1000 Classic) along the horizontal plane. The slices were then placed in a custom-made holding chamber containing high-sucrose cutting solution and incubated at 34° C. for 35 minutes. Then, the slices were transferred to another chamber containing artificial cerebral spinal fluid (aCSF) and incubated at the same temperature for 25 minutes. Following the incubation period, the brain slices were kept at room temperature. 95% O₂/5% CO₂ gas was continually bubbled into all solutions the brain slices were kept in. The cutting solution contained (in mM) 82.70 NaCl, 23.81 NaHCO₃, 2.41 KCl, 2.65 Na₂HPO₄, 14.53 MgCl₂, 0.64 CaCl₂, 23.70 Glucose and 71.19 Sucrose. The aCSF solution used during slice incubation and experiments contained (in mM): 124.98 NaCl, 23.01 NaHCO₃, 2.50 KCl, 2.36 Na₂HPO₄, 0.43 MgCl₂, 0.26 CaCl₂, and 25 Glucose. The osmolarity of the solution was 300-310 mOsm, adjusted with sucrose. All experiments were performed at room temperature.

Validation of Device Via Intracellular Calcium Response:

In order to determine the intracellular calcium response of the brain slice, Fura-2/AM (acetoxymethyl ester) (Biotium) was used. A modified version of Beierlein et al. Fura-2 loading protocol was used to prepare the brain slices for imaging. After finishing the aCSF incubation period, the hippocampal brain slices were stained with Fura-2/AM and incubated at room temperature for 60 minutes before imaging. Due to the long incubation period, a customized microfluidic device was used to oxygenate the brain slices with (21% O₂/5% CO₂) which was found to enhance cellular uptake of dye. Images used to measure the calcium response were obtained from the CA1 area of the hippocampus by measuring the Fura-2 fluorescence emission at 510 nm using a fluorescent inverted microscope (Olympus IX71). The ratiometric data was obtained by exciting the samples with 340/380 nm wavelengths using the image acquisition and analysis software MetaFluor Imaging System (Universal Imaging Corp.). For statistical analysis, the ratiometric data (340 nm intensity divided by 380 nm intensity) were converted to percent change in fluorescence by dividing the ratios obtained from each image by the average intensity ratio during the baseline-recording period (initial 5 minute period) and multiplying the result by 100; the pictures were acquired using the 10× objective. The procedure used to validate the device using the optical sensor was replicated here.

Multiple Oxygen Conditions on the Same Slice:

As previously described, another embodiment of the microfluidic device 10 of FIGS. 1A-1C is depicted in FIG. 4A. This other embodiment is essentially identical to the previous device except that instead of one microfluidic channel 42 (FIG. 1B) supplying the oxygen to the entire brain slice, there are several independent microchannels 142a, 142b, 142c (FIG. 4A) that can supply different regions of the slice with different oxygen concentrations. As with the previous device, this device docks with a common perfusion chamber 12 (Warner Instruments, RC-26GPL). The walls dividing each individual channel are 0.3 mm wide and allow complete oxygen independence from one channel to the next. This device is designed to allow the hippocampal region to be divided in 2 sections: the CA1 area can be placed on top of one channel, while the dentate gyrus can be placed on a separate channel. This concept was validated in much the same way that the first microfluidic device 10 was validated.

To simulate a stroke, one of the brain slices was placed in the device 14, in such a way that the dentate gyrus was on top of one of the channels, while the CA1 area was on top of a separate area. Then, the CA1 and the dentate gyrus were imaged. During this experiment, the CA1 area was exposed to different oxygen concentrations (0%, 95% O₂), while the rest of the slice was experiencing a constant oxygen environment (95% O₂). In a similar experiment, the Fura response, as well as the oxygen concentration, was measured at multiple positions across the channels. Using this information, the spatial resolution of the microfluidic device was determined.

Statistical Analysis:

Experiments involving animal tissue were performed on a minimum of 3 brain slices obtained from 3 different animals for a total of 9 individual data sets. Experiments not involving animal tissues were repeated a minimum of three times. Graphs show the average value with the error bars representing standard deviation.

Characterization of the Microfluidic Add-on:

To expose the brain tissue to a better controlled hypoxic environment, and as previously explained, a microfluidic add-on consisting of 4 independent parts (FIG. 1) was developed: a glass slide for support, a microfluidic channel that delivers the oxygen (250 μm thick), a thin membrane that allows oxygen diffusion (100 μm thick), and a commercially available perfusion chamber (Warner Instruments, RC-26GPL). Even though the microfluidic add-on and the perfusion method are both capable of delivering a supply of oxygen to the brain slice (FIG. 2A), the device allows more complete temporal control over a hypoxic insult to the tissue and is able to reach greater differences in oxygen concentration when compared to the perfusion method. In the literature, the time used for the hypoxic insult is not consistent from study to study, but as expected, a longer hypoxic period creates a greater deoxygenation.

The device 10 is capable of creating a hypoxic environment in less than four minutes which is faster than the previously published time of 10 minutes, and is able to revert back to its initial settings in the same amount of time, four minutes, compared to perfusion, which requires over eight minutes to equilibrate at a fluid flow rate that is compatible with electrophysiology. The device 10 is capable of reaching a level of hypoxia of 2% oxygen after an insult lasting 10 minutes while the perfusion method can only achieve 12% oxygen. However, one of the objectives of this study is to deliver a hypoxic stimulus in a time scale relevant to biological conditions. Considering how a hypoxic stimulus as short as 5 minutes can produce lasting damage to neuronal cells, it was decided to use a hypoxic stimulus lasting 4 minutes for the rest of the experiments. Using this time scale, the device 10 is also capable of achieving a level of hypoxia of 9% as compared to the perfusion method, which was only able to achieve 22% (FIG. 2B).

By eliminating the need for perfusion-driven oxygenation/deoxygenation, some of the problems inherent to this method can be avoided. Some of those problems include bubble formation resulting from switching between fluids, pulsations in the fluid level in the perfusion chamber (using both peristaltic pumps and gravity drip feed), and depending on the flow rate, a shear stress experienced by the tissue that can lead to mechanical instability. Some of these problems can be avoided if a slower flow rate is used; however, this would lead to a bigger lag in the time response than is already seen when switching between fluids (FIG. 2A) due to the dead volume in the slice chamber.

Constant Oxygen Environment:

Common methods use only perfusion to oxygenate a brain slice. Because one side of the slice faces the glass bottom of the chamber, the end result is lower oxygen condition in the middle of the slice when compared to the outer edges of the slice. While some newer methods modify the perfusion chamber in such a way as to elevate the slice in an attempt to have fluid flowing above and below the slice, however, even with this modification, an oxygen gradient within the slice is still created. By measuring the oxygen concentrations inside the brain slice at various depths (FIG. 2C), the device 10 of the present disclosure creates a noticeable gradient as the diffusion distance increased. This gradient moved in the opposite direction as the gradient produced by the perfusion method. By using the perfusion method and the device 10, a relatively uniform physiological oxygen environment is created in the brain slice. Even though the microfluidic device 10 does not create a uniform physiological oxygen environment without the help from fluid perfusion, it is capable of producing a uniform hypoxic environment throughout the entire slice with only an 8% difference from the bottom to the top of the slice, while perfusion creates a gradient of over 20% from top to bottom (FIG. 2D). These data suggest that the device 10 is both capable and efficient at producing hypoxic insults on the brain tissue in a well-controlled manner superior to current methods.

Fura-2 imaging of the hippocampus: Fluorescent calcium indicators have allowed neuroscientists to use calcium as a quantitative factor to relate oxygen deficiency to neuronal viability. The response was measured using this technique because it demonstrates the spatial control of oxygen that is able to be imposed on the brain slice. Using the microfluidic device 10 to control oxygen, the calcium response in the neurons from the CA1 area of the hippocampus was imaged to determine the relationship between neuronal function and hypoxia. The hippocampus's role in memory formation and the fact that it is particularly sensitive to oxygen level changes are well documented. Among the different areas of the hippocampus, the CA1 area is the most vulnerable to hypoxic events, followed by the dentate gyrus that also suffers neuronal damage. During a hypoxic event, overactivation of glutamate receptors allow a massive amount of calcium into the cell, which leads to a cascade of events that if not resolved, ultimately leads to cell death. Therefore, an increase in intracellular calcium levels is one indicator of a neuron experiencing a hypoxic insult. Using the ratiometric calcium indicator Fura-2, it is possible to quantify the extent of the intracellular calcium level increase. When the Fura-2 molecule binds to calcium, the ratio (340/380) intensity increases.

To image the transient calcium levels, Fura-2 AM was bath loaded into the neuronal cells of the hippocampal area. Slices were exposed to a hypoxic insult mediated either by the microfluidic device (FIG. 3A) or by the perfusion method (FIG. 3D). All of the images were taken with 10× magnification and 2 sample images taken before and during the hypoxic insult are shown in FIGS. 3B and 3C. Even though both methods were able to deliver the hypoxic stimuli and a response was obtained for each, the microfluidic device generates the more controlled stimuli. The device's Fura response is constrained to the four minutes in which the deoxygenation occurred, while the perfusion's Fura response extended for a period close to ten minutes, long after the deoxygenation should have stopped. Moreover, the peak intensity change response to hypoxia using our device was twice what we obtained with perfusion.

Spatial control over the oxygenated region: The plurality of channels 142 was used to allow multiple oxygen concentrations to affect different parts of the brain slice simultaneously (FIG. 4A, 4B). As described previously, the spatial control of oxygen levels was validated using calcium imaging combined with direct measurement of oxygen. FIG. 4C shows results when we imposed 95% oxygen and 0% oxygen in adjacent channels. The oxygen measurements show a steep change from one microfluidic channel 142a to an adjacent microfluidic channel 142b, a change mirrored by the Fura-2 calcium signal change. From the data, it is shown how a distance of 100 μm away from the channel wall, in either direction, is enough to produce a different and independent calcium response. Taking in consideration the 0.3 mm wall and the 100 μm distance away from the wall, it is shown that the diffusion or microfluidic device 14 can spatially deliver oxygen to tissues with a resolution of 500 μm. To further illustrate spatial control of oxygenation, the Fura response of the dentate gyrus and the CA1 area while each area was exposed to a different oxygen environment (FIG. 5A) were recorded. In this case, CA1 was subjected to a hypoxic insult and dentate gyrus was exposed to 95% oxygen. As expected, the CA1 area showed a distinct increase in Fura intensity signifying the increase in intracellular calcium (FIG. 5B, 5D), while the dentate gyrus did not exhibit any change in calcium concentration (FIG. 5C, 5E). This demonstrates the ability to deliver hypoxic stimuli with microscale precision and on time scales similar to in vivo stroke events.

Precise delivery of fluids including neuroactive chemicals to the acute brain slice preparation using patterned microfluidic substrates was previously demonstrated. But control of the neurochemical environment in acute brain slice physiology experiments implies the ability to control gases as well—most obviously oxygen. Spatiotemporal manipulation of the oxygen tension in a brain slice has not been practical using current technology. With our add-on microfluidic oxygenation device 10, the spatial oxygenation conditions of subregions of the brain slice can be adjusted quickly and precisely using microfluidic channels 34, 134 and a gas permeable membrane 32. In this case, it was chosen to study the dentate gyrus and the CA1 area of the hippocampus, and thus created the device 10 with 0.3 mm channel walls. Channel walls of this width are small enough to allow the two hippocampus' subregions to be imaged separately. At the same time, the device 10 preserves the ability to maintain open access from above the perfusion chamber 12 for electrophysiology and imaging tools. However, if smaller subregions were of interest, current microfluidic technology would make it feasible to create a microfluidic device with channel walls as small as 50 μm. If smaller walls are used, the height of the channel would need to be further reduced which would result in higher pressures in the channel due to the increased resistance and this would increase membrane deflection and possibly force gas bubbles through which would be problematic.

By manipulating oxygen to the slice both through the PDMS membrane 32 and via the bathing aCSF, the brain slice can be more homogeneously oxygenated or deoxygenated as compared with traditional bath chambers 12 by exposing both sides of the slice to the desired environment. The ability to more fully oxygenate the slice is an important goal, and one that has inspired several microfluidic devices. However, based on the oxygen concentration inside the tissue that we measured, the microfluidic device 10 alone is capable of delivering a hypoxic insult to the cells throughout the depth of the 350 μm thick brain slice without any external perfusion. Even if measurements from the top of the slice are needed, as is the case when using electrophysiology tools, a physiologically relevant hypoxic insult to the tissue was able to be implemented. Also, since the oxygen is flowing across the microchannel 42 or microchannels 142 and diffusing throughout the PDMS membrane 32, manipulation of the gases does not disturb the slice mechanically as in previous efforts.

The microfluidic device 10 has many possible applications as a physiology tool for neuroscience or for other similar tissue preparations. Along with the ability to create a more homogeneous oxygen environment throughout the brain slice, the brain slice is able to be subjected to hypoxic insults at controllable rates and at defined locations within the slice. As a proof of concept, it has been demonstrated that the device 14 could deoxygenate the CA1 area of the hippocampus while keeping the dentate gyrus completely unaffected. Stroke research is a prime candidate to take advantage of the ability to precisely control the spatiotemporal oxygen environment in an acute brain slice preparation. But the possibilities extend to any condition involving pathological oxygen conditions. For example, it is known that in obstructive sleep apnea, intermittent hypoxia affects the hippocampus' role in learning and memory and where the CA1 and the dentate gyrus areas are affected differently. Furthermore, with the ability to control the oxygen environment more precisely and more easily, it might be reasonable to begin to explore whether decades of brain slice work under what amounts to hyperoxygenation are good models of the physiological brain.

Of course, the potential of this device is not limited to stroke research or neuroscience. Hyperoxygenation research could also take advantage of this new technology. Cyclic oxygenation is a common event throughout the body with muscle, kidney, and cancer cells being an example. Thanks to the permeability of PDMS to gases such as hydrogen, nitrogen, helium, methane, and carbon dioxide, several other studies can be accomplished using this device 10 as a way to expose different tissues to different gases.

Thus, a novel microfluidic add-on to a commercially available perfusion chamber 12 is demonstrated with the ability to spatially and temporally control the oxygen environment throughout a brain slice. Oxygen concentration recordings and ratiometric imaging experiments are performed to demonstrate that the diffusion device can oxygenate and deoxygenate the brain slice better than perfusion alone. Microchannels 42, 142 on the microfluidic channel substrate 34, 134 make it possible for the diffusion device to spatially deliver oxygen to tissues with a resolution of 500 micrometers. Even though the microfluidic add-on was demonstrated solely on brain slices, it is reasonable to expect studies using other tissues to take advantage of this technology. Ultimately, the microfluidic add-on presented here will undoubtedly lead to higher fidelity of brain slice experiments and could be generalized to any thin tissue slice preparations.

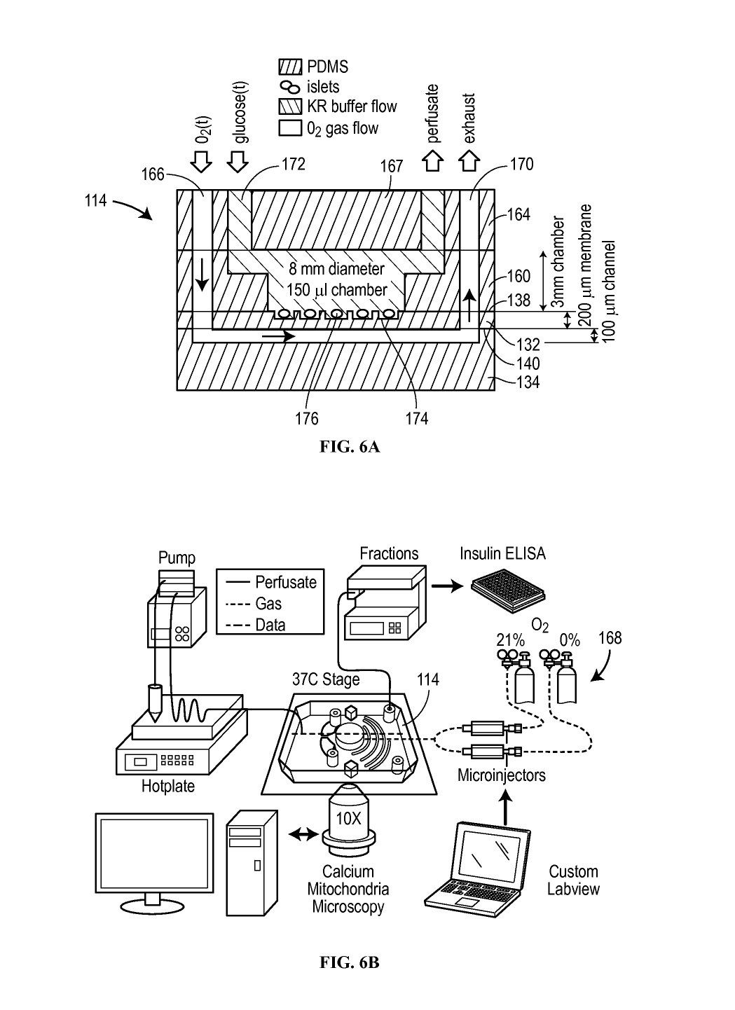

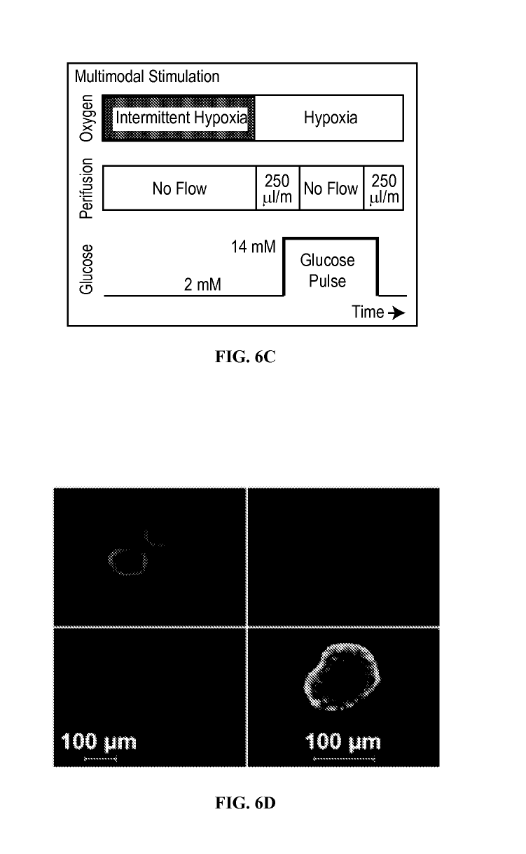

Referring now to FIGS. 6-10, another example of a microfluidic device 114 according to the present disclosure is illustrated. Generally, the microfluidic device 114 includes a multimodal islet hypoxia or reduced oxygen platform for modulating a gas environment of islets or clusters of pancreatic cells. Referring now to FIG. 6A, the microfluidic device 114 includes a gas permeable membrane 132 and a microfluidic channel 134, such that the gas permeable membrane 132 is disposed on the microfluidic channel 134 and includes a top surface 138 and a bottom surface 140. More specifically, the bottom surface 140 of the gas permeable membrane 132 contacts and is bonded to a top surface of the microfluidic channel 134. A chamber 160 is disposed on the top surface 138 of the gas permeable membrane 132, and a gasket 164 is disposed on a top surface of the chamber 140, as illustrated in FIG. 6A. A lid 167 may also be disposed within a portion of the gasket 164, as further illustrated in FIG. 6A.

The microfluidic device 114 of FIG. 6A further includes a first gas supply port 166 connectable to a first supply source 168 (FIG. 6B) and disposed through each of the microfluidic channel 134, the gas permeable membrane 132, the chamber 160 and the gasket 164. The microfluidic device 114 further includes an outlet or exhaust port 170 that is likewise disposed through each of the microfluidic channel 134, the gas permeable membrane 132, the chamber 160 and the gasket 164. The microfluidic device 114 further includes a second supply port 172 connectable to a second supply source and disposed through each of the chamber 160 and the gasket 164.

The microfluidic channel 134 delivers a first medium, such as oxygen, from the first supply port 166 to the bottom surface 140 of the membrane 132 disposed on the microfluidic channel 134 through the microfluidic channel 134. In addition, the chamber 160 delivers a second medium, such as glucose, from the second supply port 172 directly to the top surface 138 of the membrane 132, allowing simultaneous first and second media stimulation of the islets within the microfluidic device 114.

More specifically, the top surface 138 of the gas permeable membrane 132 may include at least one or a plurality of microwells 174, and the biological material, such as islets 176 in one example, is disposed within the microwells 174, as illustrated in FIG. 6A. Oxygen diffusion through the bottom surface 140 of the gas permeable membrane 132 provides direct delivery of oxygen to the islets 176.

At least one or more of the microfluidic channel 134, the gas permeable membrane 132, the chamber 160 and the gasket 164 of the microfluidic device 114 is constructed of polymethylsiloxane (PDMS), as described in more detail below. In addition, the gas permeable membrane 132 has a thickness of about 50 μm to 200 μm, and the microfluidic channel 134 has a thickness of about 50 μm to 100 μm. In the same example, the chamber 160 has a thickness of about 2.5 mm to 3.1 mm, a diameter of about 7 mm to 8.5 mm, and a volume of about 140 μl to 160 μl. More specifically, in one example, the chamber thickness is 3.0 mm, the diameter is 8 mm and the volume is 150 μl.

FIG. 6B is a schematic of microscope-based multimodal platform. FIG. 6C is chart illustrating the simultaneous control of oxygen, flow, and glucose stimulation at the islets that the microfluidic device 114 can effect. FIG. 6D shows multiple staining of islets 176 disposed within the microwells 174 of the membrane 132. More specifically, FURA 340 nm is in blue; FURA 380 nm is in green; Rhodamine is in red; and composite live-dead assay (calcein AM, ethidium bromide) is in green and red, respectively.

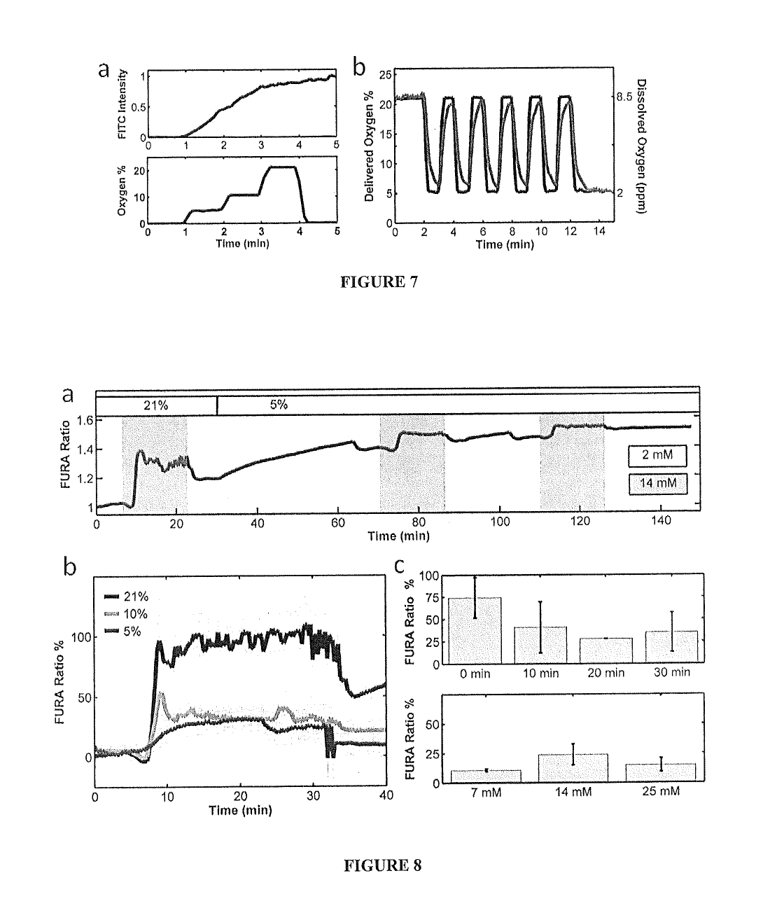

FIG. 7 provides graphs showing simultaneous oxygen and glucose stimulations results in the microfluidic device 114 of FIG. 6A. For example, the top graph of FIG. 7a shows that chamber introduction of FITC stabilizes within 3 minutes of mixing time, and the bottom graph of FIG. 7a shows that gas control provides stable delivery of 5, 10, and 21% oxygen at the membrane 134. FIG. 7b shows that cycling of oxygen between 5-21% is possible with 1 minute period as measured both at the gas delivery (diffused) and surface of microwells (dissolved), for example.

FIG. 8 provides graphs illustrating the response of islets 176 to hypoxia or reduced oxygen. More specifically, FIG. 8a is a representative overview of islet calcium response showing depressed pulse for hypoxic pulse after 30 min. 5% exposure, compared to normoxic pulse. Subsequent hypoxic pulse continues to depress response. FIG. 8b is an overlaid comparison of mean normoxic, 10%, and 5% hypoxic pulse under 14 mM glucose stimulation, showing impairments in magnitude and phase information at 5%. The top graph of FIG. 8c shows longer exposure to 5% hypoxia returns more impairment to pulse height, and the bottom graph of FIG. 8c shows various concentrations of 7, 14, and 25 mM cannot resuscitate hypoxic response. Error bars represent standard deviations.

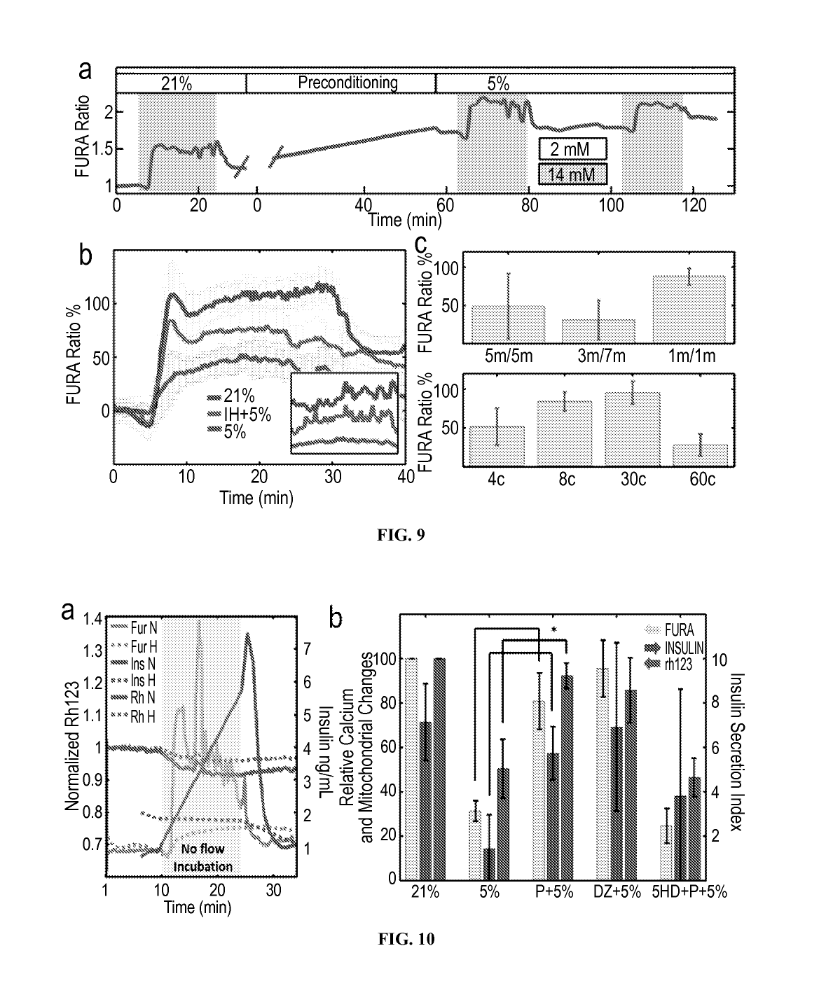

FIGS. 9A-9C provide graphs showing that IH preconditioned islets have improved response to hypoxia. More specifically, the graph of FIG. 9A is a representative overview of preconditioning using IH, totaling 30 minute exposure at 5%, showing enhanced hypoxic response compared to normoxic pulse (same islet batch in a previous experiment). 10 more minute of subsequent hypoxia still retains preconditioning. The graph of FIG. 9B is an overlaid comparison of mean normoxic, hypoxic, and preconditioned hypoxic response. Inset shows representative traces with recovery of oscillation behavior. The top graph of FIG. 9C shows that decreasing the period of cycling improves both the response magnitude as well as consistency, and the bottom graph of FIG. 9C shows that increasing the number of IH cycle improves the response. However, beyond 60 c this protocol is not optimal. Error bars represents standard deviations.

To enable simultaneous islet IH and glucose stimulations, a multi-layered microfluidic platform based on our previous islet perifusion (vs. physiological perfusion) and cell-culture oxygen gradient devices was built, combining proven islet immobilization with direct oxygen control in a stimulation sandwich, as shown in FIG. 6A. Furthermore, as the gas is continuously delivered via computerized microinjectors (FIG. 6B), the oxygen concentration is modulated from 21% to close to 0% digitally with transient times less than 60 s, as shown in FIG. 7. The dynamic oxygen and glucose microphysiology controls at the microscope allows a real-time multimodal protocol that would not be possible or extraordinarily cumbersome using standard hypoxic chambers. Using this platform, calcium, mitochondria, and insulin parameters can be monitored, allowing the investigation of: 1) quantitative hypoxia vs. islet GSM; 2) IH as a preconditioning for hypoxia; and 3) multi-parametric dynamics of IH mechanism in islets.

Simultaneous Glucose and Oxygen Stimulation:

The multilayered microfluidic platform or the microfluidic device 114 integrates both glucose and oxygen stimulations, with aqueous glucose control in the top, and oxygen manipulation in the bottom layer as shown in FIG. 6A. After dye-incubated islets 176 are loaded into the microwells 174 via the aqueous microchannels, krebs ringer buffer with basal 2 mM glucose is perfused by a peristaltic pump as shown in FIG. 6B. Stimulatory glucose concentrations—i.e. 7, 14, or 25 mM—can be subsequently introduced into the microwelled chamber 160, providing uniform concentration within 3 minutes as shown FIG. 7A. For oxygen control, a pair of labview-controlled microinjectors mixes two gas inputs proportionally, 0% and 21% O₂, each at 2 psi with 5% CO₂ and balanced with N2, to deliver oxygen concentrations of 0, 5, 10 or 21% at the microwelled membrane 132 where the islets 176 are resting. Similar to recent techniques [64-66], gas diffusion through this 200 μm polydimethylsiloxane (PDMS) membrane 132 provides direct delivery of oxygen to the islets 176, in contrast with standard hypoxic chamber that requires circulating gas to cross millimeter thicknesses of bulk aqueous medium. Note the diffusion time is inversely proportional to square of the thickness, resulting in equilibration times in seconds for the device 114 compared to hours with hypoxic chambers.

Additionally, the computerized gas control can modulate intermittent hypoxia between 5 and 21%, but other conditions can be generated based on the microinjector modulations. The limit of this method yields approximately 2 minute cycles as shown in FIG. 7B. Note that the oxygen sensor (not shown) has a delay time of 15-20 s in gas and 30-45 s in aqueous media, thus actual transient performance may be faster than shown. Gas fraction of 5% oxygen is a standard used for hypoxia in cardiac myocytes and other tissue systems. The ability to vary with this range allows us to better quantify hypoxia and IH parameters at the ex vivo level with a precision previously not possible.

Islet Glucose Calcium Response is Depressed by Hypoxia:

To quantify their response to hypoxia, FURA-incubated islets were loaded into the multimodal device and exposed to 21%, 10%, and 5% oxygen while monitoring their calcium responses. In the GSIR mechanism, the calcium response correlates with insulin secretion, thus a stimulatory glucose pulse will result in a pulse in intracellular calcium. Under 21% oxygen flow, normoxic for ex vivo islets, the calcium responded with standard bi-phasic profile with magnitudes 1.3 times above their baseline, as shown in FIG. 8A. Note that subsequent hypoxic conditions were normalized against this normoxic pulse. When the islets were exposed to 5% hypoxia for 30 minutes (under zero flow), their calcium response lost both pulse magnitude as well as phase information. Additional incubation of 10 minutes hypoxia followed by a second pulse of glucose did not improve the response. Quantitatively, the pulse height was depressed below 50% of the normoxic pulse, for both 10% and 5% hypoxia. Qualitatively, 5% hypoxia showed softened phase 0 calcium dip, rounded phase 1 overshoot, with suppressed phase 2 oscillations compared to both 10% and normoxic profiles, as shown in FIG. 8B.

The exposure time that islets were subjected to 5% hypoxia was further investigated. At 0 minute or without prior exposure, the magnitude of phase 2, not counting the overshoot, was depressed to 75% of the original pulse. After 10 minutes of exposure, this dropped to below 50%. Beyond 20 and 30 minutes the hypoxic magnitude settled around 30%. With 20 minutes of exposure at 5%, all three glucose concentrations investigated; 7, 14, and 25 mM; resulted in similar depressed pulse magnitude as shown in FIG. 8c. In summary, prominent hypoxic response occurred at 5% oxygen concentration and the effects could not be compensated with increased glucose stimulation concentrations.

IH Improves Islet Glucose Calcium Response Under Hypoxia:

To improve islet's response under hypoxia, islets were pre-exposed to IH generated via the microinjectors, as a preconditioning to hypoxia, as shown in FIG. 9A. IH (1 m/1 m cycles, 60 min total) improved islet response magnitude to 80% that of the normoxic pulse even while operating under hypoxia, as shown in FIG. 9B. Additional incubation of 10 minutes hypoxia did not reverse the response back into a hypoxia, as shown in FIG. 9A. Thus, exposures to intermittent hypoxia and continuous hypoxia were not additive and suggested different mechanisms of affecting glucose-insulin response.

The timing and cycling parameters of IH were characterized in order to optimize the preconditioning of islets. Shortening the hypoxic cycling to 1 m/1 m, as shown in FIG. 9C, provided the best pulse magnitude. When the period was fixed at 1 m/1 m and the total number of IH cycles was increased from 4 to 30, there was also an improvement in magnitude as well. However, when the cycling was increased to 60, the preconditioning benefit was removed. This limit of preconditioning duration (>2.5 hours) could be a result of the Krebs buffer perfusate in the microfluidics as compared to culture medium environments, or a competition between total hypoxic exposure and IH cycling.

Insulin Secretion is in Turn Improved by Preconditioning Via Mitochondrial KATP Channels:

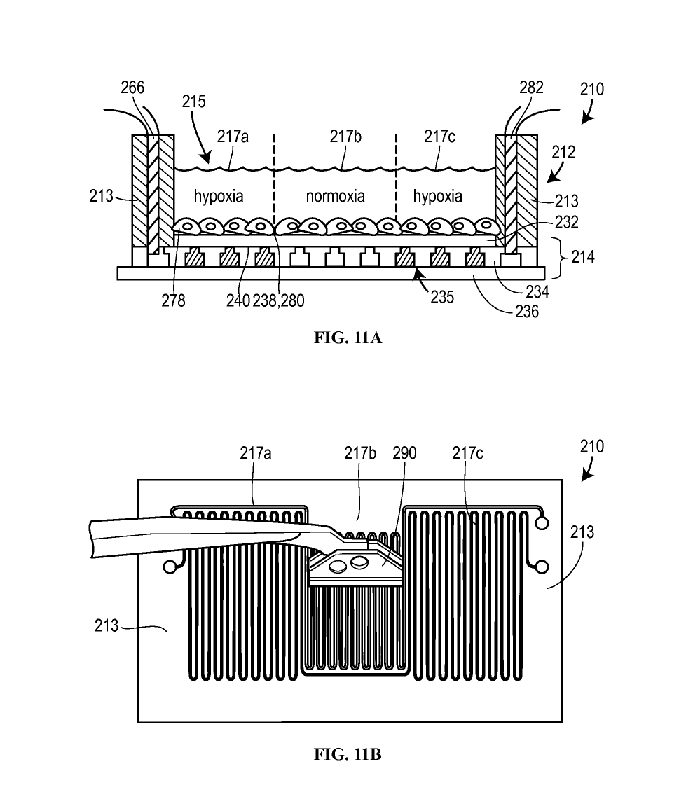

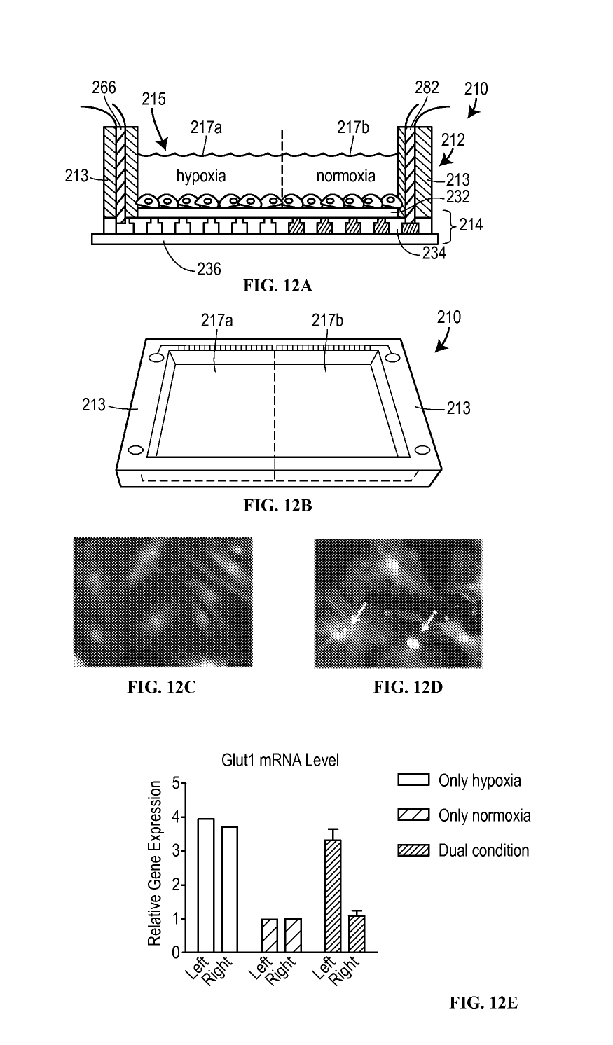

The effects of hypoxia and intermittent hypoxia preconditioning on calcium were paralleled by changes in insulin secretion and mitochondrial potential, as measured by off-chip insulin ELISA and Rhodamine 123 fluorescence, respectively. Insulin fractions were collected at 1 minute intervals during flow, with a stop-flow incubation period during the glucose exposure, as shown in FIG. 10A. The hypoxic pulse rendered insulin response down to basal levels (even drifting below baseline) while preconditioning improved insulin to 85% of normoxia while exposed to hypoxia. Note the stop-flow has a tendency to show spikes in the initial insulin samples after resumption of flow as shown in FIG. 10A. However, all insulin samples were collected in the same manner to account for this issue. Mitochondria hyperpolarization, monitored with Rhodamine uptake and self-quenching, was improved with IH preconditioning compared to hypoxia exposure alone. Preconditioning also showed sensitization of hyperpolarization with faster response than normoxic pulses. Statistical significance, as indicated in FIG. 10B, were based on p<0.05 with.

The observed benefit in hyperpolarization, suggestive of mitochondrial KATP channel involvement in IH, was investigated using KATP channel blocker 5-hydroxydecanoic acid (5HD) and opener diazoxide. Islets were incubated in 100 μM diazoxide for 10 minutes and washed in Krebs buffer for 10 minutes prior to hypoxia and glucose stimulation. Diazoxide incubation provided preconditioning benefits to islets under 5% hypoxia. Conversely, islets treated with IH (1 m/1 m cycle, 60 min total) and 100 μM 5HD simultaneously failed to be preconditioned and showed typical hypoxic response (supplementary FIG. 9 for FURA profile). Both mitochondrial hyperpolarization and insulin secretion were improved with diazoxide compared to IH+5HD, as shown in FIG. 10B. Though not statistically significant, diazoxide had higher average calcium and insulin response while its mitochondrial hyperpolarization was less than that of IH preconditioning alone. This suggests IH preconditioning is better at enhancing mitochondrial response rather than solely altering calcium-dependent insulin secretion.

Hypoxic exposure to islets has been demonstrated ex vivo to suppress insulin secretion. Evidence shows that 5% oxygen exposure represents full hypoxia compared to 10%, and specific calcium dynamic shown in this study corroborates the finding. One distinction that needs to be made is that physiological oxygenation reduces PO₂ to 40 mmHg in native pancreas, while isolated islets are exposed to 21% or 150 mmHg as their normoxic level. However, both reduction of oxygen to 5% ex vivo and correspondingly, transplant to hepatic portal vein at 5-15 mmHg both have strong hypoxic effects. Furthermore, our dynamic platform illustrated more than just lowered calcium response amplitudes, but also the reduction in phase features corresponding to calcium sequestration in ER, overshoot, and oscillatory rhythm associated with plasma membrane KATP channel, possibly due to decreased ATP in anaerobic glycolysis. Other studies have only reported total insulin secretion without dynamics, compared to our in situ hypoxic measurements.

In addition to quantifying hypoxia, the first ex vivo intermittent hypoxia preconditioning for pancreatic islets using dynamic microfluidic oxygen controls is demostrated. By optimizing cycling period down to 2 minutes with a maximum duration of 60 minutes, islets were preconditioned to produce calcium and insulin responses that are within 80-85% of the normoxic responses. In comparison, IH applied to rat myocardium at five 12 min cycles (6%/21% oxygen) showed protection against ischemia-reperfusion injury up to 24 hour after treatment. Furthermore, myocardium IH protocols (1 minute cycles) showed preference for longer treatment durations, with 4 hours IH having smaller infarction sizes than 30 minutes IH protocols. Consistent with myocardium, islet preconditioning demonstrated here is also optimal with shorter minute-scaled cycling for hour-long durations.

The demonstrated Islet preconditioning is compatible with current understanding of mitoKATP preconditioning. Mitochondrial swelling and enhanced ATP efficiency, membrane hyperpolarization dependent mitochondrial Ca2+ overloading, and reactive oxygen species regulation are the main proposed mechanisms for mitoKATP channel preconditioning. Mitochondrial hyperpolarization data in this study is consistent with the second mechanistic view. Not only does IH preconditioned mitochondria hyperpolarize more than hypoxic conditions, the results also show a faster hyperpolarization. Furthermore, 5HD introduction selectively blocked mitoKATP channels and consistently showed both decoupling of preconditioned insulin response as well as decreased mitochondrial hyperpolarization. Interestingly, sulfonylurea, a class of antidiabetic drugs, acts on membrane KATP channels to increase insulin secretion. Membrane KATP, and mitoKATP channels, are both involved in preconditioning. However, the mitoKATP channel alone can block preconditioning, while the membrane KATP, required for insulin secretion, is left untouched in this study. The multimodal platform allows specific control of the mitoKATP preconditioning both pharmacologically and metabolically (i.e. 5HD and IH) while simultaneously probing the glucose-insulin response provided by normal membrane KATP function.

The IH provided by the multimodal platform enabled islet preconditioning as well as investigations of preconditioning mechanisms via both gas phase and aqueous pharmacological agents. However, the platform has several disadvantages. As the perfusate is not oxygen-controlled, hypoxic incubation can only be conducted while the flow is stopped, and resumption of flow disrupts oxygen concentration at the islets. Insulin transients during the stop-flow are also lost. Future on-chip integration of gas-controlled perfusate channels would enable stable oxygen even during dynamic glucose stimulations while maintaining the benefits of rapid membrane-diffused oxygen to the islets. Nonetheless, the platform was able to provide the first report of IH preconditioned islets with improved insulin secretion under hypoxia. These results suggests further details in mitochondrial potential and K⁺/Ca²⁺ are required to clarify the mechanisms of both hypoxic and preconditioned islet glucose-insulin response. The platform coupled with multimodal stimulation provides just the tool to enable these future studies.

A direct application of islet IH preconditioning is improving islet function prior to transplantation. The ability to improve islet function not only addresses the hypoxia conditions but can also optimize the number of isolated islets and required transplant size. Future demonstration of preconditioning in animal and clinical models would mean that two out of three major issues are solved for islet transplantation, paving the way for broader acceptance of the method. Beyond islet preconditioning, the multimodal platform describe here can be applied to study general IH and hypoxia conditioning for various transplant sensitive systems, embroid bodies, and other microtissues. Further knowledge of KATP channel function could explain why preconditioning works so well in cardiac myocytes, and used to benefit other tissues. In addition, mechanisms in lipotoxicity and glucosetoxicity are suspected to be dependent on reactive oxygen species and high oxygen metabolism. Both aspects can be investigated by modulating gas phase oxygen in addition to aqueous oxygen scavengers using the multimodal platform. Lastly, many development/regenerative mechanisms in stem-cell differentiation and cell-cell interactions depend on oxygen gradients. The ground work laid out by the multimodal oxygen platform can be adapted to many of these challenging unanswered questions.

### Methods

Islet Intermittent Hypoxia Platform Fabrication:

The platform is comprised of three microfluidic and one blank gasket PDMS layers. The microfluidic layers—gas microchannel, microwell membrane, and glucose channel layers from bottom to top, respectively, were fabricated using standard polydimethylsiloxane soft lithography. First, microfeatures were patterned in SU8 masters using standard lithography. 100 μm thick SU8-2100 was used for the gas channels and microwell masters while 700 μm (two 350 μm) layer SU8-2150 was used for the glucose microchannel master. Then, degassed PDMS is spun onto the microwell master at 900 rpm twice to form the 200 μm gas-permeable membrane layer. Degassed PDMS was also molded over the remaining masters to form all four layers. All layers were cured on the hotplate at 80° C. for 3 hours. Appropriate cross-layer ports on the microwell membrane, glucose microchannel, and blank gasket layers were punched, including an 8 mm diameter chamber on the glucose layer. All four layers were bonded sequentially from bottom up with 30 s of exposure from an ETP plasma surface treatment device (ETP, Inc). Lastly, the device was leak-tested with simultaneous loading of water and compressed air at 2 psi.

Microinjector Gas Mixing:

Two precision microdispensing nozzles were acquired from The Lee Company and connected with their output end in a T-shaped mixer tubed to the microfluidic device. Inputs of 0 and 21% oxygen with 5% CO₂ were connected to each input end of the nozzels, with pressure adjusted to 2 psi equally. This microinjector system was driven by two 20V servos (packaged by The Lee Co.) controlled with a USB-based National Instrument TTL controller. A laptop ran the labview script written to control both nozzle openings. Cycling the nozzle on and off at proportional ratios created a mixing that can deliver calibrated concentrations between 0-21% in 1% increments. The custom labview program also provided up to five consecutive stages of concentrations for the purpose of modulating intermittent hypoxia.

Islet Isolation and Preparation:

C57BL/6 mice were sacrificed as islet donors for the murine islet experiments. 5 ml collagenase P solution at 0.375 mg/ml in HBSS was prepared for each animal and kept on ice. The animal was euthanized with CO₂ and 3% isoflurane followed by cervical dislocation to ensure no discomfort. The animal was disinfected with 70% ethanol before making a V-incision at the genital area and moving the bowel to clearly expose the bile duct. The ampulla on the surface of duodenum was clamped with a hemostat. The pancreas was then distended (inflated) through the bile duct with a 30-gauge needle with 5 ml syringe containing 2 ml of cold collagenase solution, starting at the gall bladder. The pancreas was then removed and placed in a 15 ml tube containing 2 ml of collagenase solution in a 37° C. water bath for 12 minutes. Then, 10 ml of cold HBSS was added to stop the digestion when 80% of the pancreas has disintegrated. The digestion was centrifuged at 284 g's (1000 r.p.m. in a Beckman J6-MI) for 30 s at 4° C. and the supernatant discarded. The pellet was resuspended in 14 ml cold HBSS and centrifuged and supernatant removed again. The islets were purified using a discontinuous Ficoll gradient density of 1.096, 1.069, and 1.037 kg/m³ and centrifugation at 640 g and 4° C. The islets were picked up from the interface between density layers 1.069 and 1.096 using a plastic transfer pipette and placed in a 50 ml conical tube containing 25 ml of cold HBSS. The islets were washed 2 more time with cold HBSS via the centrifuge. Afterwards, the islet pellet was resuspend in 10 ml RPMI-1640 containing 10% FBS, penicillin, streptomycin, and 20 mM HEPES and transferred into a Petri-dish and place in a humidified incubator (37° C., 5% CO2). All islets were cultured for 1 day post-isolation prior to use in experiments.

Krebs Buffer, FURA, Rhodamine 123, and Off Chip Insulin ELISA:

All fluids perfused through the MIIH device were prepared in Krebs Ringer bicarbonate buffer: 129 mM NaCl, 5 mM NaHCO₃, 4.7 mM KCl, 1.2 mM KH₂PO₄, 1 mM CaCl₂.2H₂O, 1.2 mM MgSO₄.7H₂O, 10 mM HEPES pH 7.35-7.40. FURA-2 AM, stock solution was prepared in DMSO and added to Krebs buffer at a final concentration of 5 μM. Rho123 stock solution was prepared in 100% ethanol and also added to Krebs buffer at 2.5 μM final concentration. The islets were incubated in 2 ml of Krebs buffer containing both FURA and Rho123 at those concentrations for 30 minutes prior to loading in the device. Using a 10 μl pipette tip the dye-incubated islets were loaded into the glucose inlet port of the microfluidic device. Subsequent pipetting from the outlet to the inlet assured smooth loading of the islets into the microwell traps. Then, 2 mM glucose Krebs buffer was perfused through the device for 5 minutes to wash the excess dye from the chamber.

Glucose and Krebs buffer flow were only introduced while washing or stimulating the islets, and flow was stopped at all other times. Insulin in the forms of effluent fraction were collected from the device 4 minutes before the stimulation arrives at the islet and 3 minutes after starting the wash flow at the end of the pulse. For reference, it takes 4-5 minutes for effluents to travel to the fraction collector. Fractions were immediately frozen at −20° C. and later thawed for 96-plate insulin ELISA measurements (Mouse Insulin, Mercodia, Sweden). The plates were read in a Biotek Synergy 2 plate reader.

Islet Loading onto the Multimodal Platform:

The number of islets loaded depends on the type of experiments: approximately 10 for calcium and rhodamine dye monitoring; approximately 20 for insulin experiments. The islets were loaded at the perifusion inlet, then perfused for 5 minutes to wash away excess dyes. Perfusate flow was provided by a Gilson Minipulse 2 peristaltic pump. Tubing carying the perfusate was heated on a 60° C. hotplate upstream of the microfluidic device. The microfluidic device was mounted on an inverted microscope with heated stage. Gas input from the microinjector was also connected at microfluidic device. Effluent from the device outlet was tubed to a Gilson 203 fraction collector, and immediately frozen after the experiment.

### Data and Statistical Analysis

An inverted Leica S6F microscope provided simultaneous FURA and Rho123 measurements. FURA was excited at 340 and 380 nm and measured at 510 nm while Rho123 was excited at 495 nm and measured at 530 nm FURA measurements were expressed as a ratio of 340 nm over 380 nm intensities. The ratiometric measurement accounts for both dye loading and photobleaching during each experiment. Furthermore, every experiment began with a standard pulse at 21% oxygen followed by pulses at variable conditions. The data was then normalized against the standard normoxic pulse to minimize variation between experiments and the results were expressed as “FURA Ratio %”. Rho123 measurement and the associated hyperpolarization drop were also analyzed similarly by normalizing against a normoxic standard. The insulin ELISA timepoints, measured in ng/mL, was expressed as an index of the maximum value over the baseline average to account for the stop flow during glucose/hypoxic incubation and also varying number of islets in the experiments.

For the FURA calcium measurements, n=4 experiments were conducted for each normoxic, hypoxic, precondition, diazoxide, and 5HD conditions. For hypoxic parameters, n=3 experiments were conducted for each of the concentration and exposure conditions. For preconditioning parameters, n=3 experiments were conducted for each of the period and cycling conditions. For the Rhodmine mitochondrial experiments, n=3 experiments were conducted for each condition. For insulin ELISA, n=3 experiments were conducted for each condition. A paired-sample t-test (2-tailed) was performed to calculate P values for the difference between the means of the hypoxia and preconditioning conditions. Error bars in each figure represent standard deviations of the total number of experiments conducted.

Referring now to FIG. 11, another assembly 210 for modulating a gas environment of a biological material, such as cultured cells, is depicted. The assembly 210 comprises an open well device 212 having a pair of sidewalls 213 and a chamber 215 disposed within the sidewalls 213, the chamber 215 having at least two regions 217. In the example open well device 212 of FIGS. 11A and 11B, the chamber 215 includes three regions 217a, 217b, and 217c, of which the middle region 217b is a normoxic or normal oxygen region, and the regions 217a and 217c flanking the middle region 217b are hypoxia or reduced oxygen regions, as explained in more detail below.

The assembly 210 further includes a microfluidic device 214 onto which the open well device 212 is disposed. The microfluidic device 214 includes a slide 236, a microfluidic channel substrate 234 disposed on the slide 236, and a gas permeable membrane 232 disposed on the microfluidic channel 234. The microfluidic channel substrate 234 includes a plurality of microchannels 235 for delivering gas or other medium to the gas permeable membrane 232, as explained in more detail below. Like the gas permeable membranes of earlier examples, the gas permeable membrane 232 of the microfluidic device 214 includes a top surface 238 and a bottom surface 240. The top surface 238 of the gas permeable membrane 232 forms a base 280 of the chamber 215 of the open well device 212 upon disposal of the open well device 212 on the microfluidic device 114, as illustrated in FIG. 11A.

The assembly 210 further includes at least one supply port 266 connected to a supply source (not shown) and disposed through each of the open well device 212 and the microfluidic device 214. A medium from the supply port 266 is delivered through the microfluidic channel substrate 234 to the bottom surface 240 of the gas permeable membrane 232 to modulate and/or control the gas environment in each region 217, e.g., 217a, 217b, and 217c, of the chamber 215. Each region 217 may be the same width as a cell scraper 290 (FIG. 11B). In addition, biological material 278, such as cultured cells, is disposed on the top surface 238 of the gas permeable membrane 232. One or more of the gas permeable membrane 232 and the microfluidic channel 234 disposed under the membrane 232 may be constructed of PDMS.

The assembly 210 may further include a second supply port 282 connectable to a second supply source (not shown) and disposed through each of the open well device 212 and the microfluidic device 214. More specifically, the first supply port 266 is disposed through one of the sidewalls 213 of the open well device 212, and the second supply 282 is then disposed through the other of the sidewalls 213, such as the right sidewall 213 of FIG. 11A. In addition, the first supply port 266 is adapted to supply one of various preselected levels of process medium to control the gas environment in one or more regions 217a, 217b and 217c of the chamber 215 of the open well device 212. Likewise, and in a similar manner, the second supply port 282 is adapted to supply the same or a different level of process medium to control the gas environment in another one of the one or more regions 217a, 217b, 217c of the chamber 215 of the open well device 214. More specifically, and as explained in more detail below, the assembly 210 may include a dual condition device in which the chamber 215 of the open well device 212 is exposed to two distinct oxygen conditions. More specifically, the plurality of microchannels 235 deliver either reduced oxygen (hypoxia) or normal oxygen (normoxa) levels to cells cultured on the gas permeable membrane 232.

Referring now to FIGS. 11B-11D, a top perspective view of the assembly 210 is illustrated in FIG. 11B, and a cell scraper 290 is illustrated as having the same width as the middle region 217b of the chamber 215 of the open well device 212. While not illustrated in FIG. 11B, the cell scraper 290 may also have the same width as either of other regions 217a, 217c flanking both sides of the central region 217b. FIG. 11C shows that HIF-1alpha staining localizes to the cell nucleus under hypoxia. FIG. 11D shows that when cells are harvested and GLUT1 mRNA is quantified, it is higher in hypoxic regions (H=Hypoxia, N=Normoxia).

In FIGS. 11A-11D, cells were cultured on a 100 μm gas permeable PDMS membrane, which is the substrate of the chamber and contains the oxygen microchannels underneath (FIG. 11). These condions were driven by the inputs and can be altered by switching input gas feeds. Mouse lung epithelial cells exposed to hypoxia had HIF-gas channels with an ability to probe cellular behavior via immunostaining and PCR. In addition, 1 alpha protein localized to the cell nuclei and increased GLUT1 mRNA expression, as expected from previous studies. These studies validate the method of O₂ delivery via buried microfluidic this device exposes cells to varying O₂ tensions in the same monolayer with intact paracrine signalling between hypoxic and normoxic regions as in vivo.

Additional detailed discussion of similar methods and apparatus may be found in co-pending and commonly assigned U.S. patent application Ser. No. 12/527,897, filed Oct. 10, 2009, the entire dislcosure of which is incoporated by reference herein.

Referring now to FIGS. 12A-12E, another embodiment of the assembly 210 is provided. The embodiment of the assembly 210 includes many of the same component parts and, therefore, the same reference numerals as the other embodiment of the assembly 210 of FIG. 11A, where indicated. Unlike the assembly 210 of FIG. 11A, however, the embodiment 210 of FIG. 12A includes only two regions 217a and 217b within the chamber 215 of the open well device 212 and is sometimes referred to as a dual condition device. More specifically, the region 217a is the hypoxia or reduced oxygen region and region 217b is the normoxia or normal oxygen region.

The assembly 210 of FIG. 12A was designed to flow oxygen through the microfluidic channel substrate 234, which, in one example, is 500 μm wide and 150 μm deep, and allow diffusion across the gas permeable membrane 232, which, in one example, is 100 μm thick. Standard lithography techniques were used for microfabrication, wherein channel features were replicated in PDMS from SU-8 masters. The microfluidic channel substrate 234 includes a first microfluidic channel network 234a having the plurality of microchannels 235 and a second microfluidic channel network 234b having microchannels 235 as well. In one example, the first microfluidic channel network 234a is 26.2 mm long×25.4 mm wide and is adjacent the equal-sized second microfluidic channel network 234b. The first and second microfluidic channels 234a and 234b are separated by a 500 μm gap. Due to the close proximity of the microfluidic channels, diffusion in the tissues or cells disposed on the gas permeable membrane 232 is rapid. In view of this structure, discrete oxygen environments can be patterned in a single continuous monolayer of cells. For example, in the dual condition assembly 210 of FIG. 12A, constant perfusion of 5% CO2 balanced nitrogen may be in one microfluidic channel network 234a and 5% CO2 balanced air may be in the adjacent network 234b, establishing a steady hypoxic region 217a and a normoxic region 217b, respectively.

In addition, and similar to FIGS. 11B-11D, a top perspective view of the assembly 210 is illustrated in FIG. 12B, with blue food dye in the hypoxic region 217a and yellow food dye in the normoxia region 217b. FIGS. 12C and 12D shows fluorescence immunostaining of nuclei (blue) and HIF-1α (green) in normoxic region (D) and the hypoxic region (E). As expected, there is nuclear localization of HIF-1α in hypoxia. In other words, HIF-1α staining localizes to the cell nucleus under hypoxia. FIG. 12F shows that when cells are harvested and GLUT1 mRNA is quantified, it is higher in hypoxic regions (H=Hypoxia, N=Normoxia).

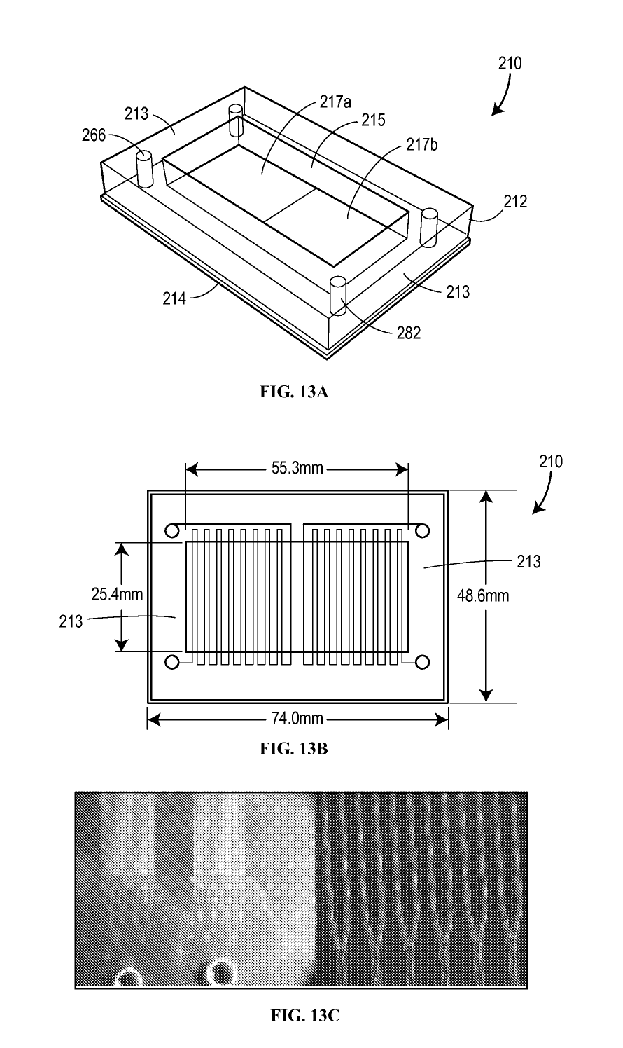

Referring now to FIGS. 13A-13C, other views of the assembly 210 of FIG. 12A are shown. More specifically, FIG. 13A illustrates a perspective view of the assembly 210, further showing the sidewalls 213 of the open well chamber 212 disposed on the microfluidic device 214. In addition, the regions 217a and 217b are shown disposed within the chamber 215 of the open well device 212 and first and second supply ports 266, 282, respectively, are also provided. FIG. 13B illustrates a top view of the assembly 210 of FIG. 13A. While various lengths and widths may be selected for the assembly 210, in one example, the length of the assembly is 74 mm and the width is 48.6 mm. In addition, in the same example, the length of the chamber 215 of the open well device 212 is 55.3 mm and the width is 25.4 mm. Of course, one of skill in art will appreciate that various other dimensions may be selected for both the length and width parameters of the assembly 210 and the chamber 215 of the open well device 212 disposed on the microfluidic device 214 to form the assembly 210. FIG. 13C shows images of embossed microfluidic channel substrate 234 having a width of 30 μm.

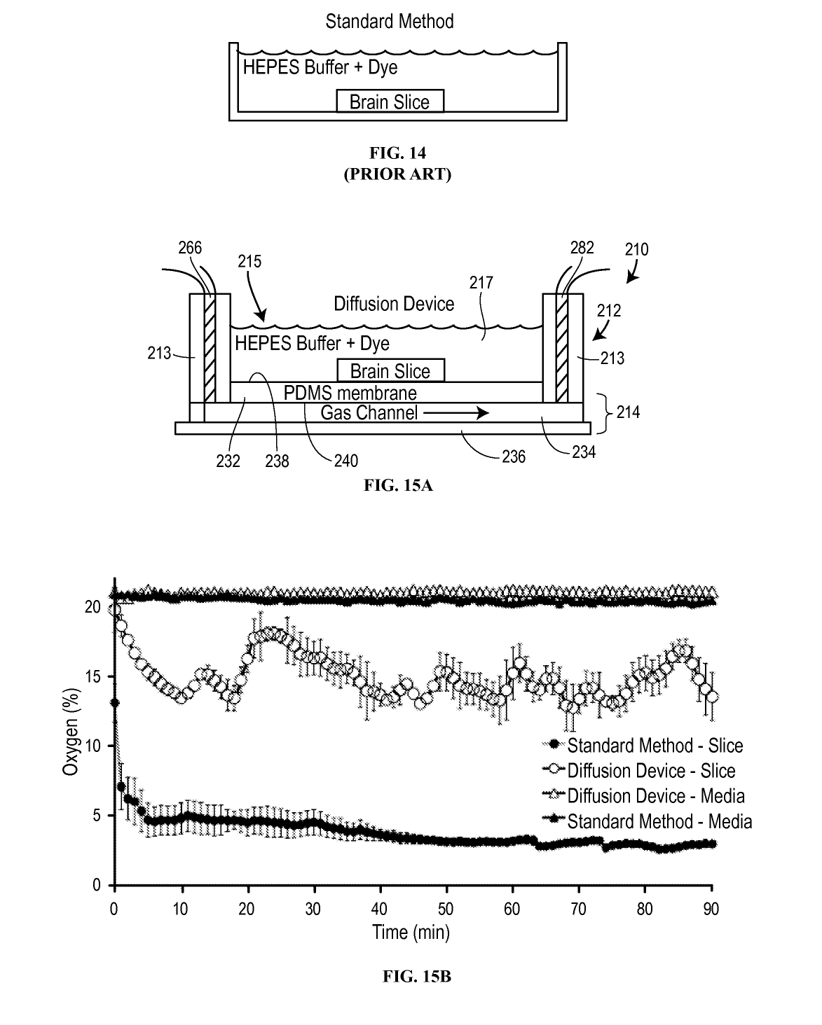

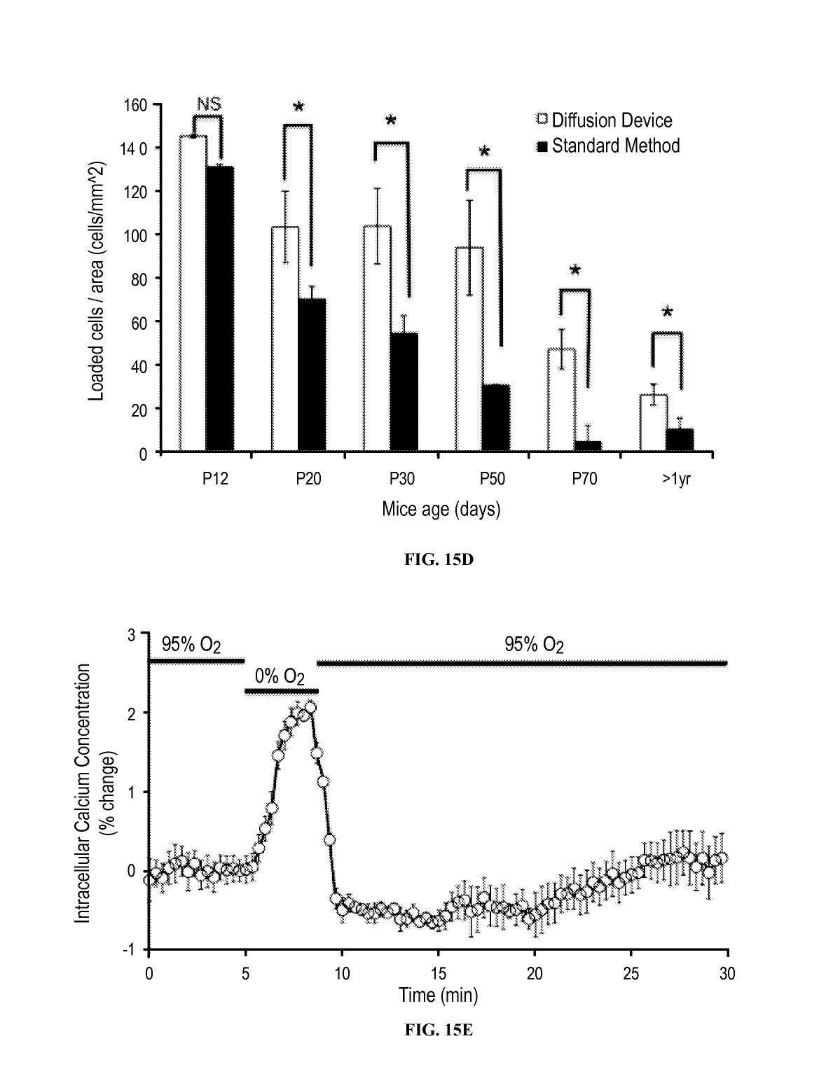

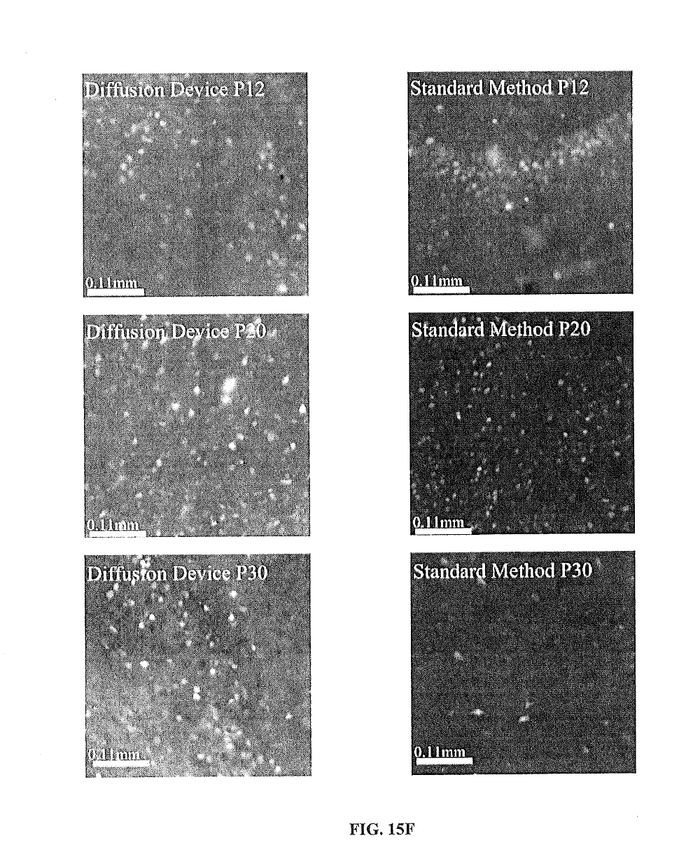

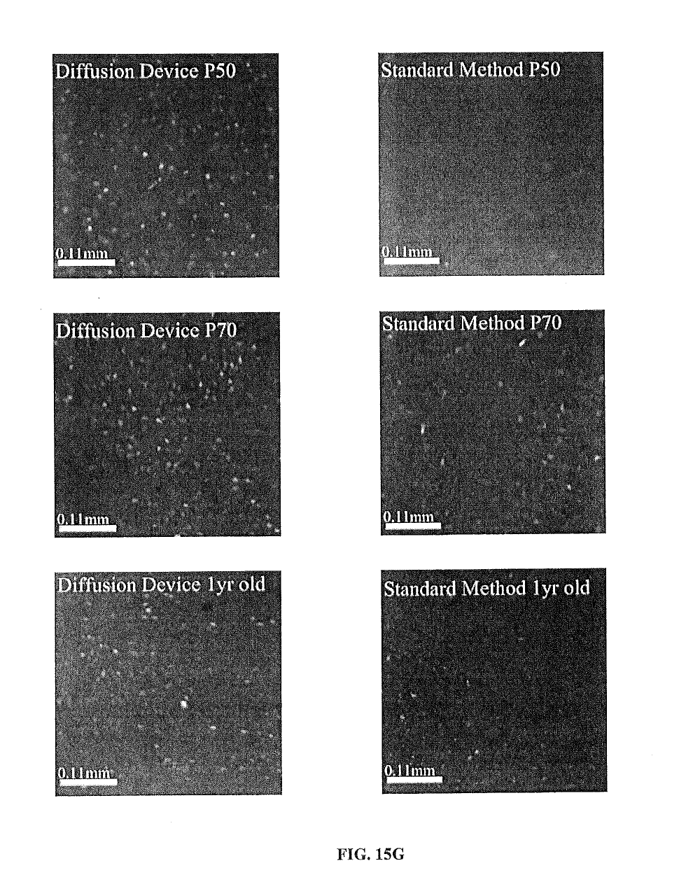

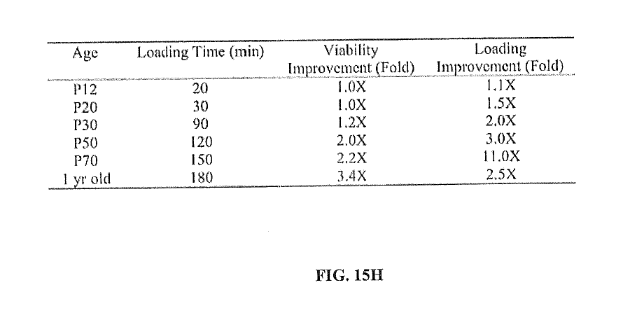

Referring now to FIGS. 14 and 15A-15H, another embodiment of an assembly 210 is provided. This embodiment of the assembly 210 again includes many of the same component parts and, therefore, the same reference numerals as the other embodiments of the assembly 210 of FIGS. 11A and 12A, where relevant. Unlike the assembly 210 of FIG. 11A that includes three regions 217a, 217b, and 217c or the assembly 210 of FIG. 12A that includes two regions 217a, 217b within the chamber 215 of the open well device 212, the embodiment 210 of FIG. 15A includes one region 217 within the chamber 215. This assembly 210 of FIG. 15A is sometimes referred to as a single condition device. More generally, the assembly 210 consists of a microfluidic device 214 having a microfluidic channel substrate 234 with a single microfluidic channel disposed beneath the gas permeable membrane 232. This assembly 210 having the single condition device may be suitable for researchers who wish to run live imaging experiments and harvest cellular material for later analysis at single conditions, for example.

In contrast, FIG. 14 illustrates the structure of a device utilizing a prior art standard method of diffusion, and FIGS. 15B-H compare various results of both the standard method of the device in FIG. 14 and the assembly 210 of FIG. 15A. More specifically, FIG. 15B illustrates oxygen concentration (%) as a function of time (min) under both the standard method of FIG. 14 and for the microfluidic device of FIG. 15A, FIG. 15C is a graph illustrating the percentage of live cells as a function of mice age (days) for both the standard method of FIG. 14 and the microfluidic device of FIG. 15A. FIG. 15D is another graph illustrating the percentage of loaded cells per area (cells/mm 2) as a function of mice age (days) for both the standard method of FIG. 14A and the microfluidic device of FIG. 15A. FIG. 15E is yet another graph illustrating the intracellular calcium concentration (% change) as a function of time for the biological material of the microfluidic device of FIG. 15A. In addition, FIGS. 15F and 15G show various magnified images of a portion of the biological material increasing in time, e.g., P12, P20, P30, for both the standard method of FIG. 14 and the microfluidic device of FIG. 15A. FIG. 15H is a table showing loading time (min), viability improvement (fold), and loading improvement (fold) for various ages of the biological material in the microfluidic device of FIG. 15A.

From the foregoing it will be appreciated that, although specific embodiments of the invention have been described herein for purposes of illustration, various modifications may be made without deviating from the spirit and scope of the invention.

What is claimed is:

1. An microfluidic device comprising:
a perfusion chamber, the perfusion chamber having a base, a bath opening in the base, a supply inlet, and an exhaust outlet;
a gas permeable membrane attached beneath the perfusion chamber, the gas permeable membrane having a first opening in registration with the supply inlet and a second opening in registration with the exhaust outlet;
a substrate attached to the gas permeable membrane, the substrate having at least one microchannel arranged for flow communication with the supply inlet and the exhaust outlet; and
a slide attached to the substrate;
wherein a gas introduced through the supply inlet is communicated to the microchannel via the first opening, and further wherein the gas permeable membrane is positioned to be exposed to the gas to communicate the gas to the bath opening.

2. The microfluidic device of claim 1, wherein the bath opening defines an area to receive biological material, the biological material comprising one or more of a brain slice or cultured cells.

3. The microfluidic device of claim 1, wherein a top surface of the gas permeable membrane defines an area to receive biological material, and wherein the microfluidic channel substrate includes a plurality of separate flow channels or microchannels, at least some of the plurality of flow channels arranged to be selectively blocked, and wherein the biological material may be exposed to different concentrations of a gas communicated through the plurality of flow channels.

4. The microfluidic device of claim 1, wherein one or both of the gas permeable membrane and the microfluidic channel substrate is constructed polydimethylsiloxane (PDMS).

5. The microfluidic device of claim 1, wherein the thickness of the membrane is about 50 μm to 200 μm.

6. The microfluidic device of claim 1, wherein the thickness of the microfluidic channel substrate is about 50 μm to 100 μm.

7. The microfluidic device of claim 1, wherein the substrate further includes a support pillar that prevents the gas permeable membrane from collapsing.

8. A microfluidic device for modulating a gas environment of a biological material, the device comprising:
a microfluidic channel;
a gas permeable membrane disposed on the microfluidic channel, the gas permeable membrane having a top surface and bottom surface;
a chamber disposed on the top surface of the gas permeable membrane;
a gasket disposed on the chamber;
a first supply port connectable to a first supply source and disposed through each of the microfluidic channel, the membrane, the chamber, and the gasket; and
a second supply port connectable to a second supply source and disposed through each of the chamber and gasket;
wherein the microfluidic channel delivers a first medium from the first supply port directly to the bottom surface of the membrane disposed on the microfluidic channel, and the chamber delivers a second medium from the second supply port directly to the top surface of the membrane, allowing simultaneous first and second media stimulation within the microfluidic device.

9. The microfluidic device of claim 8, wherein at least one or more of the microfluidic channel, the gas permeable membrane, the chamber, and the gasket is constructed polydimethylsiloxane (PDMS).

10. The microfluidic device of claim 8, wherein the top surface of the membrane includes a plurality of microwells, and biological material rests or is disposed within the plurality of microwells.

11. The microfluidic device of claim 10, wherein the biological material comprises islets or clusters of pancreatic cells.

12. The microfluidic device of claim 11, wherein the first medium is oxygen and the second medium is glucose, such that the device allows simultaneous oxygen and glucose stimulation of the islets within the microfluidic device.

13. The microfluidic device of claim 12, wherein oxygen diffusion through the bottom surface of the membrane provides direct delivery of oxygen to the islets.

14. The microfluidic device of claim 8, wherein the gas permeable membrane has a thickness of about 50 μm to 200 μm.

15. The microfluidic device of claim 8, wherein the microfluidic channel has a thickness of about 50 μm to 100 μm.

16. The microfluidic device of claim 8, wherein the chamber has a thickness of about 2.5 mm to 3.1 mm, a diameter of about 7 mm to 8.5 mm, and a volume of about 140 μl to 160 μl.

17. An assembly for modulating a gas environment of a biological material, the assembly comprising:
an open well device having a pair of sidewalls and a chamber with at least two regions disposed between the sidewalls;
a microfluidic device onto which the open well device is disposed, the microfluidic device having a slide, a microfluidic channel substrate disposed on the slide and having a plurality of microchannels, and a gas permeable membrane disposed on the microfluidic channel substrate, the gas permeable membrane having a top surface and a bottom surface, the top surface of the gas permeable membrane forming a base of the chamber of the open well device upon disposal of the open well device on the microfluidic device; and
at least one supply port connected to a supply source and disposed through each of the open well device and the microfluidic device;
wherein a medium from the supply port is delivered through the microfluidic channel substrate to a bottom surface of the gas permeable membrane to modulate the gas environment of each region of the chamber.

18. The assembly of claim 17, wherein the chamber includes at least three regions, the regions including a central normoxic or normal oxygen region flanked by two hypoxic or reduced oxygen regions.

19. The assembly of claim 17, wherein each region is the same width as a cell scraper allowing simple cell extraction.

20. The assembly of claim 17, wherein biological material is cultured on the top surface of the gas permeable membrane, and the biological material is one or more of a cluster of cells or a brain slice.

21. The assembly of claim 17, wherein one or both of the membrane and microfluidic channel disposed under the membrane is constructed of polydimethylsiloxane (PDMS).

22. The assembly of claim 17, further comprising a second supply port connectable to a second supply source and disposed through each of the open well device and the microfluidic device.

23. The assembly of claim 22, wherein the first supply port is disposed through one of the sidewalls of the pair of sidewalls of the open well device and the second supply port is disposed through the other of the sidewalls of the pair of sidewalls of the open well device, the first supply port adapted to supply one of various preselected levels of a process medium to control the gas environment in one or more regions of the chamber of the open well device, and the second supply port adapted to supply the same or different levels of process medium to control the gas environment in another one of the one or more regions of the chamber of the open well device.

24. The assembly of claim 17, wherein the medium from the supply port is delivered through the microfluidic channel substrate to a bottom surface of the gas permeable membrane to modulate the gas environment of each region of the chamber via diffusion.

25. The assembly of claim 17, wherein the device is a dual condition device in which the chamber of the open well device is exposed to two distinct oxygen conditions.

26. The assembly of claim 17, wherein the open well device is a fluid reservoir.

27. A method of modulating an oxygen environment for biological material, the method comprising:
attaching a bottom surface of a gas permeable membrane to at least one of a microfluidic channel or a microfluidic channel substrate, the at least one of a microfluidic channel or a microfluidic channel substrate disposed below the gas permeable membrane;
attaching a chamber to a top surface of gas permeable membrane;
placing biological material on the top surface of the gas permeable membrane below the chamber;
delivering a first medium from a supply port in communication with a supply source and disposed through one of the microfluidic channel or the microfluidic channel substrate and the chamber directly to the bottom surface of the membrane to modulate the gas environment of the biological material.

28. The method of claim 27, further comprising delivering a second medium from a second supply port in communication with a second supply source and disposed through the chamber directly to the bottom surface of the membrane.

29. The method of claim 27, further comprising delivering a second medium from a second supply port in communication with a second supply source and disposed through an interior surface of the chamber directly to the top surface of the membrane.

30. The method of claim 29, wherein the first medium is oxygen and the second medium is glucose, and the method further comprises simultaneously stimulating glucose and oxygen in biological material disposed on the top surface of the membrane.

31. The method of claim 30, further comprising covering one or both of the gas permeable membrane and the at least one of the microfluidic channel or the microfluidic channel substrate with polydimethylsiloxane (PDMS).

32. An microfluidic device comprising:
a gas permeable membrane;
a substrate attached to the gas permeable membrane, the substrate having at least one microchannel or microfluidic channel; and
a supply port connectable to a supply source and arranged for flow communication with the at least one microchannel or microfluidic channel;
wherein a medium introduced through the supply port is communicated to the at least one microchannel or microfluidic channel, and further wherein the gas permeable membrane is positioned to be exposed to the medium via the at least one microchannel or microfluidic channel.
